(19) United States
(12) Patent Application Publication
(10) Pub. No.: US 2026/0185984 A1
(43) Pub. Date: Jul. 2, 2026

(54) POTENCY ASSAY FOR CD34+ CELLS

(71) Applicant: CELLPROTHERA SAS, Mulhouse (FR)

(72) Inventors: Christine VIGNON, Mulhouse (FR); Ibon GARITAONANDIA, Mulhouse (FR); Guillaume TREBUCHET, Mulhouse (FR); Matthieu DE KALBERMATTEN, Mulhouse (FR)

(73) Assignee: CELLPROTHERA SAS, Mulhouse (FR)

(21) Appl. No.: 19/130,734

(22) PCT Filed: Nov. 17, 2023
(86) PCT No.: PCT/EP2023/082283

§ 371 Date: May 16, 2025

(30) Foreign Application Priority Data
Nov. 18, 2022 (EP) 22306707.5

(51) Int. Cl. G01N 33/50; A61K 35/28; C12N 5/0789; G01N 33/543

(52) U.S. Cl. CPC G01N 33/5094; A61K 35/28; C12N 5/0647; G01N 33/54386; C12N 2500/02; C12N 2501/125; C12N 2501/145; C12N 2501/2303; C12N 2501/2306; C12N 2501/727; G01N 2333/475; G01N 2474/00

(57) ABSTRACT

The disclosure relates to methods for the selection of CD34+ cells, cells selected by the methods and therapeutic uses for those cells. More, specifically the disclosure relates to CD34+ cells which are selected based on a level of VEGF expression and/or cellular and/or exosomal microRNA expression. The disclosure therefore provides an in vitro method for selecting CD34+ cells based on determining the amount of VEGF and/or identifying miRNAs expressed by a population of CD34+ cells, and selecting CD34+ cells accordingly.

### CROSS REFERENCE TO RELATED APPLICATION(S)

This application is a 35 U.S.C. § 371 National Stage of International Patent Application No. PCT/EP2023/082283, filed Nov. 17, 2023, which claims priority to European Patent Application No. 22306707.5, filed Nov. 18, 2022, each of of which is incorporated by reference in its entirety for all purposes.

### SEQUENCE LISTING

The present application includes a Sequence Listing filed in ST.26 format. The Sequence Listing, created on Nov. 17, 2023, is named “B2520219WO_ST26 Sequence Listing.xml” and is 23,356 bytes in size. The information in the electronic format of the Sequence Listing is part of the present application and is incorporated herein by reference in its entirety.

### TECHNICAL FIELD

The disclosure relates to methods for the selection of CD34+ cells, cells selected by the methods and therapeutic uses for those cells. More, specifically the disclosure relates to CD34+ cells which are selected based on a level of VEGF expression and/or cellular and/or exosomal microRNA expression.

### BACKGROUND

CD34 is a cell surface marker used to identify and isolate hematopoietic stem/progenitor cells (HSPCs). CD34+ cells are commonly isolated from blood samples using immunomagnetic techniques. CD34+ cells can differentiate into all types of blood cells as well as endothelial cells.

Intracardiac delivery of autologous peripheral blood-derived CD34+ stem cells (CD34+ cells) mobilized by granulocyte-colony stimulating factor (G-CSF) and collected by leukapheresis after myocardial infarction, has been shown to structurally and functionally repair the damaged myocardial area (Pasquet S, Sovalat H, Hénon P et al.). An automated device (StemXpand®) that allows stem cell expansion after G-CSF mobilisation has been developed and shown to provide CD34+ cells numbers at least equivalent to those collected during leukapheresis (Saucourt, Vogt, Merlin et al.). Characteristics (CD34+− cell numbers, purity/impurity profile, and viability) and safety (sterility, pyrogen, and mycoplasma content) of the expanded cells has been assessed and the functionality of expanded cells (eCD34+) has been demonstrated in an in vivo preclinical study in rats.

Advanced therapy medicinal products (ATMPs) are medicines for human use that are based on genes, tissues or cells. Such products must be evaluated for potency on a batch by batch basis as required by legislation in both the United States and Europe. The potency assay should relate to the mechanism of action and correlate to the desired clinical outcomes.

There is, therefore, a need for a simple in vitro potency assay for CD34+ cells that can confirm the therapeutic efficacy of said cells in clinical applications.

### SUMMARY OF THE INVENTION

The present invention provides an in vitro method for selecting CD34+ cells comprising:
(i) determining the amount of VEGF expressed by a population of CD34+ cells, and
(ii) selecting CD34+ cells based on the amount of VEGF expressed by the population.

The CD34+ cells may be selected if the amount of VEGF expressed by the cells is at least about 1 fg/cell.

The invention further provides an in vitro method for selecting CD34+ cells comprising:
(i) detecting one or more microRNAs expressed by a population of CD34+ cells and/or contained in their exosomes, and
(ii) selecting CD34+ cells based on the one or more microRNAs expressed by the population and/or contained in their exosomes.

The invention further provides an in vitro method for selecting CD34+ cells comprising:
(i) detecting one or more microRNAs expressed by a population of CD34+ cells and/or contained in their exosomes and determining the amount of VEGF expressed by a population of CD34+ cells, and
(ii) selecting CD34+ cells based on the one or more detected microRNAs and the amount of VEGF expressed.

The CD34+ cells may be selected if the expression of one or more or each of miR126, miR130a, miR21, miR26a, miR378a, miR146a, miR199a, miR590 and miR133a is detected.

The CD34+ cells may be selected if the expression of one or more or each of miR126, miR130a, miR21, miR26a, and miR378a is detected.

The CD34+ cells may be selected if the expression of one or more or each of miR21, miR26a, and miR378a is detected.

The CD34+ cells may be selected if the expression of miR146a and/or miR21 is detected.

The CD34+ cells may be selected if the expression of miR199a and/or miR590 is detected.

The CD34+ cells may be human CD34+ cells.

The CD34+ cells may be autologous or allogeneic CD34+ cells.

The methods of the invention may further comprise expanding a population of CD34+ cells before and/or after steps (i) or (ii).

The cells may be expanded for 9 days.

The expanding may comprise one or more of;
expanding at 37° C.,
expanding in a 5% CO₂ controlled atmosphere, and/or
expanding in a culture medium comprising cytokines such as interleukin 6 (IL6), interleukin 3 (IL3), Stem Cell Factor, ThromboPoietin, and/or Fms-Like Tyrosin kinase 3 Ligand.

The amount of VEGF may be determined by ELISA or automated ELISA (ELLA).

The amount of VEGF may be determined by mass spectrometry.

The amount of VEGF may be determined by radioimmunoassay.

The amount of VEGF may be determined by multiplex assay.

The methods of the invention may further comprise collecting, centrifuging and/or purifying the selected cells, optionally wherein the purifying is by immunoselection.

The cells may be for use in treatment.

The invention further provides an isolated population of CD34+ cells selected by the methods of the invention.

The invention further provides an isolated population of CD34+ cells selected by a method disclosed herein for use in therapy.

The invention further provides an isolated population of CD34+ cells selected by a method disclosed herein for use in treatment of myocardial infarction.

### BRIEF DESCRIPTION OF THE DRAWINGS

FIG. 1: Plate layout for VEGF concentration assay

FIG. 2a-c: VEGF standard curves

FIG. 3: VEGF concentration in supernatant after 9 days of CD34+ cell expansion (Assay 1)

FIG. 4a: VEGF concentration in supernatant after 9 days of patient CD34+ cell expansion (Assay 1)

FIG. 4b: VEGF concentration in supernatant after 9 days of healthy donor CD34+ cell expansion (Assay 1)

FIG. 5a: VEFG concentration and CD34+ cells count after a 9 day expansion of CD34+ cells from Acute Myocardial Infarction (AMI) patients; VEGF concentration curves obtained from supernatant (Assay 1)

FIG. 5b: VEGF concentration and CD34+ cells count after a 9 day expansion of CD34+ cells from AMI patients; graph of correlation (Assay 1)

FIG. 6: VEGF concentration in supernatant after 9 days of CD34+ cell expansion (Assay 2)

FIG. 7a: VEGF concentration in supernatant after 9 days of patient CD34+ cell expansion (Assay 2)

FIG. 7b: VEGF concentration in supernatant after) 9 days of healthy donor CD34+ cell expansion (Assay 2)

FIG. 8a: VEFG concentration and CD34+ cells count after a 9 day expansion of CD34+ cells from AMI patients; VEGF concentration curves obtained from supernatant (Assay 2)

FIG. 8b: VEGF concentration and CD34+ cells count after a 9 day expansion of CD34+ cells from AMI patients; graph of correlation (Assay 2)

FIG. 9: VEGF concentration in supernatant after 9 days of CD34+ cell expansion (Assay 3) FIG. 10a: VEGF concentration in supernatant after 9 days of patient CD34+ cell expansion (Assay 3)

FIG. 10b: VEGF concentration in supernatant after 9 days of healthy donor CD34+ cell expansion (Assay 3)

FIG. 11a: VEFG concentration and CD34+ cells count after a 9 day expansion of CD34+ cells from AMI patients; VEGF concentration curves obtained from supernatant (Assay 3)

FIG. 11b: VEGF concentration and CD34+ cells count after a 9 day expansion of CD34+ cells from AMI patients; graph of correlation (Assay 3)

FIG. 12: VEGF concentration in supernatant after 9 days of CD34+ cell expansion (Average of assays 1, 2 and 3)

FIG. 13a: VEGF concentration in supernatant after 9 days of patient CD34+ cell expansion (Average of assays 1, 2 and 3)

FIG. 13b: VEGF concentration in supernatant after 9 days of healthy donor CD34+ cell expansion (Average of assays 1, 2 and 3)

FIG. 14a: VEFG concentration and CD34+ cells count after a 9 day expansion of CD34+ cells from AMI patients; VEGF concentration curves obtained from supernatant (Average of assays 1, 2 and 3)

FIG. 14b: VEGF concentration and CD34+ cells count after a 9 day expansion of CD34+ cells from AMI patients; graph of correlation (Average of assays 1, 2 and 3)

FIG. 15: VEGF concentration in supernatant collected after expansion of CD34+ cells from AMI patients (curves obtained for assays 1, 2 and 3)

FIG. 16a: Expression of CD63/CD81 and CD34 in CD45+CD34+ (ProtheraCytes®) cell-derived exosomes (positive fraction) from various patients. From left to right, the bars correspond to FHD_4, FHD_5, FHD_6, FHD_8 and P_081.

FIG. 16b: Expression of CD63/CD81 and CD34 in CD45+CD34-cell-derived exosomes (negative fraction) from various patients. From left to right, the bars correspond to FHD_4, FHD_5, FHD_6, FHD_8 and P_081.

FIG. 17: Housekeeping genes (miR-103a, let7a-5p and U6) analysed for stability

FIG. 18: Proangiogenic miRNAs (miRNA126; miRNA130a; miRNA378a) in ProtheraCytes® exosomes from AMI patient (P081). Pt F+ Cell=Positive Fraction_Cells; Pt F+ Cell=Patient_Positive Fraction_Exosomes; Pt F− Cell=Patient_Negative Fraction_Cells; Pt F− Cell=Patient_Negative Fraction_Exosomes

FIG. 19A: Proangiogenic miRNAs (miRNA126; miRNA130a; miRNA378a) in ProtheraCytes® exosomes from FHD (C4.1; MicroRNA study 4; Essai_Cytokines comparison-1-K0321-12120). Pt F+ Cell=Positive Fraction_Cells; Pt F+ Cell=Patient_Positive Fraction_Exosomes; Pt F− Cell=Patient_Negative Fraction_Cells; Pt F− Cell=Patient_Negative Fraction_Exosomes

FIG. 19B: Proangiogenic miRNAs (miRNA126; miRNA130a; miRNA378a) in ProtheraCytes® exosomes from FHD (C5.1; MicroRNA study 5; Essai_Cytokines comparison-2-K0321-12120). Pt F+ Cell=Positive Fraction_Cells; Pt F+ Cell=Patient_Positive Fraction_Exosomes; Pt F− Cell=Patient_Negative Fraction_Cells; Pt F− Cell=Patient_Negative Fraction_Exosomes

FIG. 19C: Proangiogenic miRNAs (miRNA126; miRNA130a; miRNA378a) in ProtheraCytes® exosomes from FHD (C6.1; MicroRNA study 6; Essai_Cytokines comparison-3-K0321-12120). Pt F+ Cell=Positive Fraction_Cells; Pt F+ Cell=Patient_Positive Fraction_Exosomes; Pt F− Cell=Patient_Negative Fraction_Cells; Pt F− Cell=Patient_Negative Fraction_Exosomes

FIG. 19D: Proangiogenic miRNAs (miRNA126; miRNA130a; miRNA378a) in ProtheraCytes® exosomes from FHD (C8.1; MicroRNA study 8; Essai_Stab_SF 279511 TOM. Pt F+ Cell=Positive Fraction_Cells; Pt F+ Cell=Patient_Positive Fraction_Exosomes; Pt F− Cell=Patient_Negative Fraction_Cells; Pt F− Cell=Patient_Negative Fraction_Exosomes

FIG. 20: Proangiogenic miRNAs (miRNA126-3p; miRNA130a-3p; miRNA378a)-3p in ProtheraCytes® exosomes. RTqPCR_Exosome from ProtheraCytes® from FHD (n=5) and AMI patient (n=1). Pt F+ Cell=Positive Fraction_Cells; Pt F+ Cell=Patient_Positive Fraction_Exosomes; Pt F− Cell=Patient_Negative Fraction_Cells; Pt F− Cell=Patient_Negative Fraction_Exosomes

FIG. 21: Expression of miRNAs analysed in CD34+ cells after 9 days of culture (ProtheraCytes®) versus exosomes from ProtheraCytes® (exosomes; nanovesicles produced by ProtheraCytes®) from 7 patients (062, 065, 066, 068, 072, 079, 081). Proangioangenic miRNAs: miR126, miR130a, miR21, miR26a, miR378a; Antiapoptotic effect of miRNAs: miR146a, miR21; miRNAs increasing proliferation of cardiomyocytes: miR199a, miR590; Antifibrotic miRNA: miR133a

FIG. 22: Comparison of the average miRNA expression obtained in the exosomes produced by ProtheraCytes® with that obtained in the ProtheraCytes® of the 7 patients analyzed (062, 065, 066, 068, 072, 079, 081).

FIG. 23a: VEGF secreted by CD34+ cells isolated from a selection of patients in the EXCELLENT clinical trial (fg/cell).

FIG. 23b: Absolute change in N-terminal prohormone of brain natriuretic peptide (NT-proBNP) (pg/mL) at 6 months following administration of CD34+ cells compared to baseline vs. level of VEGF secretion/cell (fg/cell) in 13 patients. Graph shows a negative correlation between VEGF secretion/cell (fg/cell) and N-terminal prohormone of brain natriuretic peptide (NT-proBNP) (pg/mL).

### DETAILED DESCRIPTION

The present inventors have shown that CD34+ cells secrete vascular endothelial growth factor (VEGF) and levels of VEGF present in cell culture supernatant following expansion closely correlate with the total number of CD34+ cells. The present inventors have also shown that CD34+ cells and CD34+ derived exosomes contain proangiogenic miRNAs (miR126, miR130a, miR378a, miR26a), antiapoptotic miRNAs (miR21 and miR146a), miRNAs promoting myocardial regeneration (miR199a and miR590) and antifibrotic miRNAs (miRNA133a).

Determination of the amount of VEGF expressed by a population of CD34+ cells and/or the miRNAs expressed by a population of CD34+ cells can therefore be used as part of a potency assay for the selection of CD34+ cells. Such a potency assay can form part of the approval process for Advanced Therapy Medicinal Products, such as the isolated CD34+ cells of the invention

The potency assay is a critical quality control measure required to determine whether expanded cells possess the characteristics that will produce the desired effect. For example, desired characteristics may be the ability to promote cardiac regeneration and revascularization. The potency assay allows for the quick measurement of potency before the batch is released for injection in the clinic.

### Definitions

“CD34+ cells” are progenitor cells that can differentiate into all types of blood cells as well as endothelial cells. CD34+ cells are mobilized from the bone marrow into the peripheral blood by the administration of hematopoietic growth factor. Total CD34+ cells represent approximately 0.5-1% of total bone marrow derived mononuclear cells. CD34+ cells comprise hematopoietic stem/progenitor cells (HSPCs) as well as endothelial progenitor cells (EPCs). HSPCs can differentiate into all blood cell types and EPCs can differentiate into endothelial cells. CD34+ cells grow in suspension cultures. CD34+ cells may also be obtained from umbilical cord blood.

“VEGF” (vascular endothelial growth factor) is a potent proangiogenic growth factor known to stimulate the formation of new blood vessels.

“ProtheraCytes®” is the trade name for the applicant's purified CD34+ cells for use in therapeutic applications. ProtheraCytes® are human autologous CD34+ cells expanded according to a GMP automated manufacturing process designed for large clinical scale production. ProtheraCytes® are registered as an ATMP-Advanced Therapy Medicinal Product-within the classification of Tissue Engineered product by the European Medicines Agency.

“StemXpand®” is the trade name for an incubator system for the automated expansion of cells, such as CD34+. The StemXpand® system is described in U.S. Pat. No. 10,676,705B2.

“StemFeed®” (Eurobio, France) is the trade name for a proprietary medium for expansion of CD34+ cells comprising basal IMDM medium, human plasma and a mix of cytokines.

“microRNAs” (miRNAs) are small non-coding RNAs that influence gene expression.

### Isolation of CD34+ Cells

The CD34+ cells may be obtained from a whole blood sample. The whole blood sample may be obtained from a donor subject. The donor subject may be a human. The donor subject may be a human in need of treatment. The donor subject may be a human in need of treatment for myocardial infarction. CD34+ cells may also be obtained from umbilical cord blood. The CD34+ cells may be autologous or allogeneic CD34+ cells.

The whole blood sample may be obtained following granulocyte-colony stimulating factor (G-CSF) mobilisation. The whole blood sample may be subjected to red blood cell sedimentation. The whole blood sample may be subjected to total nuclear cell isolation. Total nuclear cell isolation may follow the gelatin method whereby the whole blood sample is mixed with a gelatin solution and hung for a period of time to facilitate red blood cell sedimentation. The red blood cells remaining in the pellet may be mixed with gelatin and hung for a second period of time. Following sedimentation, the supernatant may be centrifuged to pellet the total nuclear cells. Following centrifugation, the CD34+ cells may be purified by immunoselection. Immunoselection may be performed by any known method, for example with the CliniMACS system (Magnetic-Activated Cell Sorting).

### Culture or Expansion of CD34+ Cells

The purified CD34+ cells be cultured or expanded before and/or after the steps of determining the amount of VEGF expressed by a population of CD34+ cells, detecting the miRNAs expressed by a population of CD34+ cells and selecting a population of CD34+ cells. The CD34+ cells may be cultured or expanded for 5 to 12 days. The CD34+ cells may be cultured or expanded for 5, 6, 7, 8, 9, 10, 11, or 12 days. The CD34+ cells may be cultured or expanded for 9 days. The CD34+ cells may be cultured or expanded at 37° C. The CD34+ cells may be cultured or expanded in a 5% CO₂ controlled atmosphere. The CD34+ cells may be cultured or expanded in a culture medium comprising cytokines such as interleukin 6 (IL6), interleukin 3 (IL3), Stem Cell Factor, ThromboPoietin, and Fms-Like Tyrosin kinase 3

Ligand at various concentrations. Cells may be cultured at any suitable concentration, for example 2.5×10⁵ cells/mL

Methods of the invention may be carried out on human autologous CD34+ cells expanded according to a GMP automated manufacturing process designed for large clinical scale production, e.g. ProtheraCytes®.

### Determination of VEGF Expression

The amount of VEGF expressed by a population of CD34+ cells may be determined by any known means, for example, by western blot, enzyme-linked immunosorbent assay (ELISA), ELLA system (Bio-Techne's automated immunoassay platform), fluorescence-linked immunosorbent assay (FLISA), competition assay, radioimmunoassay, lateral flow immunoassay, flowthrough immunoassay, electrochemiluminescent assay, nephelometric-based assays, turbidometric-based assay, or fluorescence activated cell sorting (FACS)-based assays. Determination of the amount of VEGF may be by mass spectrometry. Determination of the amount of VEGF may be by radioimmunoassay. Preferably, determination of the amount of VEGF is by ELISA. For example, determining the amount of VEGF expressed by a population of CD34+ cells may involve one or more of the following:
collecting supernatant from CD34+ cell cultures;
storing the supernatant;
measuring VEGF using an ELISA kit e.g. QuantiGlo ELISA Kit (R&D Systems, MN, USA) according to the manufacturer's instructions, for example with the SpectraMax L (Molecular Devices, San Jose, CA USA) or Simple Plex Cartridge Kit containing VEGF-A for use with Human Cell Supernatant with ELLA Protein Simple system (Bio-Techne).

A negative control may be used, for example, a culture medium such as StemFeed® medium. A positive control may be used, for example, Immunoassay Control Set 732 for Human VEGF (R&D Systems). The amount of VEGF expressed by a population of CD34+ cells may be determined by measuring the concentration of VEGF released by the cells into a cell culture medium. The amount of VEGF expressed by a population of CD34+ cells may be determined by measuring the concentration of VEGF contained in CD34+ cell derived exosomes. The CD34+ cell culture or a portion thereof may be centrifuged and the concentration of VEGF present in the supernatant determined. For example, approximately 50 ml of supernatant may be obtained and frozen as smaller aliquots. Typically, 50 μL of sample per well in the ELISA assay may be used.

The CD34+ cells may be selected according to the method, if the amount of VEGF expressed by the cells in a culture medium is an amount indicative of biological and/or therapeutic activity or efficacy. For example, CD34+ cells may be selected according to the method, if the amount of VEGF expressed by the cells in a culture medium is at least about 1, 5, 10, 20, 25, 50, 75, 100, 150, 155, 160, 165, 170, 175, 180, 185, 190, 195 or 200 μg/ml. The CD34+ cells may be selected according to the method, if the amount of VEGF present in the cell culture medium or supernatant of a CD34+ cell culture is at least 25 μg/ml. The CD34+ cells may be selected according to the method, if the amount of VEGF present in the cell culture medium or supernatant of a CD34+ cell culture is at least 50 μg/ml. The CD34+ cells may be selected according to the method, if the amount of VEGF present in the cell culture medium or supernatant of a CD34+ cell culture is at least 150 μg/ml. If the concentration of VEGF contained in CD34+ cell derived exosomes is measured, selection may be based on any threshold value disclosed herein.

The amount of VEGF expressed by a population of CD34+ cells may be determined by measuring an amount of VEGF expressed as an amount per cell. The CD34+ cell culture or a portion thereof may be centrifuged and the amount of VEGF present in the supernatant determined. The number of CD34+ cells may be counted by any known method, for example flow cytometry, and the amount of VEGF may be expressed as an amount per cell. The CD34+ cells may be selected if the amount of VEGF expressed per cell is at least about 0.5 fg, at least about 1 fg, at least about 1.5 fg, at least about 2 fg, at least about 2.5 fg, or at least about 3 fg. The CD34+ cells may be selected if the amount of VEGF expressed per cell is at least about 1 fg.

The CD34+ cells may be selected for use in treatment.

Detection of miRNA Expression

A sample of CD34+ cells may be taken for miRNA analysis following the culturing or expansion described above.

The expression of miRNA in a population of CD34+ cells may be detected by measuring miRNA expression in CD34+ cells and/or in CD34+ cell derived exosomes. miRNA may be isolated from exosomes and/or cells and measured.

Exosomes may be purified by centrifugation of CD34+ cells to remove the cells and cell debris. The resulting supernatant may then be centrifuged again to pellet the exosomes. For example, 50 ml of culture supernatant may be collected, and may be frozen into smaller aliquots. Smaller volumes, such as, 200 μL of sample, may be used for extracting miRNA from the exosomes. The miRNA may then be extracted from the exosomes by any known method, such as the use of a commercially available miRNA extraction kit. miRNA may be extracted from CD34+ cells by any known method, such as the use of a commercially available miRNA extraction kit. One example of a commercially available miRNA extraction kit is the Qiagen® miRNeasy® kit.

The miRNAs may be detected and/or quantified by any known method. For example, the miRNAs may be detected and quantified by Quantitative real-time PCR (RT-qPCR), digital PCR, microarray, luminescence with QuantiGene® miRNA (Affymetrix, Life-Technologies) and/or high-throughput small RNA-sequencing. One example of a commercially available kit for miRNA detection and quantification is the Qiagen® miRCURY LNA miRNA PCR kit. Suitable primers available from Qiagen® include YP00204230 (miR-21-5p), YP00206023 (miR-26a-5p), YP00204227 (miR-126-3p), YP002046658 (miR-130a-3p), YP00204788 (miR-133a-3p), YP00204688 (miR146a-5p), YP00204536 (miR-199a-3p), YP00205946 (miR-378a-3p), and YP00205448 (miR-590-3p). The qPCR data may be normalized to miR-let7a-5p (YP00205727) values. Relative miRNA expressions may be calculated using the 2^{-ΔΔCt} method.

The miRNAs disclosed herein may be detected by, for example, detecting either a 3p and/or a 5p miRNA strand. For example, the miRNAs disclosed herein may be detected via any of the following strands:

```
(SEQ ID NO: 1)
miR126-3p UCGUACCGUGAGUAAUAAUGCG
  (SEQ ID NO: 10)
miR126-5p CAUUAUUACUUUUGGUACGCG
  (SEQ ID NO: 2)
miR130a-3p CAGUGCAAUGUUAAAAGGGCAU
  (SEQ ID NO: 11)
miR130a-5p GCUCUUUUCACAUUGUGCUACU
  (SEQ ID NO: 16)
miR21-3p CAACACCAGUCGAUGGGCUGU
  (SEQ ID NO: 3)
miR21-5p UAGCUUAUCAGACUGAUGUUGA
  (SEQ ID NO: 17)
miR26a-3p CCUAUUCUUGGUUACUUGCACG
  (SEQ ID NO: 4)
miR26a-5p UUCAAGUAAUCCAGGAUAGGCU
  (SEQ ID NO: 5)
miR378a-3p ACUGGACUUGGAGUCAGAAGGC
  (SEQ ID NO: 12)
miR378a-5p CCUCCUGACUCCAGGUCCUGUGU
  (SEQ ID NO: 18)
miR146a-3p CCUCUGAAAUUCAGUUCUUCAG
  (SEQ ID NO: 6)
miR146a-5p UGAGAACUGAAUUCCAUGGGUU
  (SEQ ID NO: 7)
miR199a-3p ACAGUAGUCUGCACAUUGGUUA
  (SEQ ID NO: 13)
miR199a-5p CCCAGUGUUCAGACUACCUGUUC
  (SEQ ID NO: 8)
miR590-3p UAAUUUUAUGUAUAAGCUAGU
  (SEQ ID NO: 14)
miR590-5p GAGCUUAUUCAUAAAAGUGCAG
  (SEQ ID NO: 9)
miR133a-3p UUUGGUCCCCUUCAACCAGCUG
(SEQ ID NO: 15)
miR133a-5p AGCUGGUAAAAUGGAACCAAAU
```

Selection of CD34+ Cells Based on miRNA Expression

The CD34+ cells may be selected if miRNA indicative of biological and/or therapeutic activity or efficacy is detected. For example, the CD34+ cells may be selected if the expression of one or more or each of miR126, miR130a, miR21, miR26a, miR378a, miR146a, miR199a, miR590 and miR133a is detected.

The CD34+ cells may be selected if the expression of one or more or each of miR126, miR130a, miR21, miR26a, and miR378a is detected.

The CD34+ cells may be selected if the expression of one or more or each of miR21, miR26a, and miR378a is detected.

The CD34+ cells may be selected if the expression of miR146a and/or miR21 is detected.

The CD34+ cells may be selected if the expression of miR199a and/or miR590 is detected.

The CD34+ cells may be selected if the expression of miR133a is detected.

The CD34+ cells may be selected for use in treatment.

Determination of VEGF Expression and Detection of miRNA Expression

The method of the invention may comprise both (a) detecting one or more microRNAs expressed by a population of CD34+ cells and/or contained in their exosomes and (b) determining the amount of VEGF expressed by a population of CD34+ cells. In this embodiment, selection of the cells is based on the one or more microRNAs and the amount of VEGF expressed by the population. Steps (a) and (b) may be carried out sequentially in any order, or in parallel. In this embodiment, the amount of VEGF expressed may be determined as described elsewhere herein. In this embodiment, microRNA may be detected as described elsewhere herein.

The CD34+ cells may be selected if (i) miRNA indicative of biological and/or therapeutic activity or efficacy is detected and (ii) VEGF expressed by the cells is an amount indicative of biological and/or therapeutic activity or efficacy.

The CD34+ cells may be selected if a VEGF threshold disclosed herein is met and if the presence of one or more microRNAs (or any combination of microRNAs) disclosed herein is detected. The CD34+ cells may be selected for use in treatment.

### Additional Processing Steps

Following the selection of CD34+ cells as described above, the CD34+ cells may undergo further processing steps, such as, for example, purification and/or immunoselection.

The CD34+ cells may be immunoselected for example using magnetic activated cell sorting. The immunoselected CD34+ cells may then be resuspended in a buffer. The buffer may be 4% albumin in saline. The buffer may be phosphate buffered saline (PBS)/2% human serum albumin (HSA).

Said processing may be to form a product suitable for use in a method of treatment. For example, ProtheraCytes® are CD34+ cells which have been processed to form a product suitable for use in a method of treatment. ProtheraCytes® are registered as an ATMP (Advanced Therapy Medicinal Product) within the classification of Tissue Engineered product by the European Medicines Agency.

### Isolated Population of Cells

The invention further provides a population of isolated CD34+ cells selected by any of the methods disclosed herein. The selected cells may have any of the properties of the CD34+ cells disclosed herein.

For example, the cells may express an amount of VEGF disclosed herein. The cells may express one or more microRNAs disclosed herein. The population of cells may be provided as a composition comprising suitable excipients. The composition may, for example, comprise PBS and/or human serum albumin.

### Methods of Treatment

Cells obtained by the method of the invention may be used in therapy. For example, the cells may be used in the treatment of myocardial infarction. The cells may be administered by intra-cardiac injection. The cells may promote a functional and structural cardiac regeneration of an ischemic lesion after myocardial infarction. Administration of cells of the invention may cause a reduction in NT-proBNP in the subject being treated.

### INCORPORATION BY REFERENCE

All documents cited herein are incorporated by reference to the fullest extent permitted by law.

### EXAMPLES

### Example 1: Obtaining CD34+ Cells

### Patient Samples, Healthy Donors and Cell-Production Centers

AMI patients and healthy male volunteers were enrolled in this study after approval by the French regulatory agency Agence Nationale de Sécurité du Médicament et des produits de santé and the regional ethics committee. All participants provided signed informed consent. Each participant first underwent daily subcutaneous (s.c.) administration of 10 μg/kg per day G-CSF (Lenograstim) for 4 days. A whole blood (WB) sample of 440 ml±10 ml was withdrawn in the morning of the fifth day by simple venous puncture and collected in a blood bag and immediately shipped at ambient temperature to the Cell Production Centre. The manufacturing process was started on the sixth day, after overnight storage of the WB sample at 4° C.-8° C. C, using the StemXpand automated integrated system and StemPack disposable kits developed by CellProthera.

### ProtheraCytes Preparation

Starting from the initial WB sample, red blood cell (RBC) sedimentation was performed for total nuclear cell (TNC) isolation using the gelatin method. Briefly, 440 ml of WB/phosphate-buffered saline 1:1 solution (PBS; Macopharma, Mouvaux, France) was mixed with 440 ml of 4% gelatin (Gelofusine, BBraun, Melsungen, Germany) in two 600-ml transfer bags, which were hung for 20 minutes to facilitate RBC sedimentation. RBCs remaining in the pellet were again mixed with 4% gelatin for a second 20-minute sedimentation period. The two supernatants were pooled and centrifuged at 400 g for 10 minutes at room temperature to pellet the TNC, from which basal (b)-CD34+ stem cells (SCs) were purified using the CliniMACS system (Magnetic-Activated Cell Sorting, Miltenyi Biotec, Bergisch Gladbach, Germany). The bag containing purified b-CD34+SC suspension or thawed frozen healthy donor (FHD) CD34+ cells (Lonza), was immediately connected to the machine kit to undergo a 9-day culture period in our proprietary StemFeed medium into the StemXpand incubator, in which the expansion steps are automatically programmed and controlled: first, predetermined volumes of StemFeed culture medium, cytokine mix (composed of interleukin [IL]6, IL3, Stem Cell Factor, ThromboPoietin, and Fms-Like Tyrosin kinase 3 Ligand at various concentrations), and the CD34+ SCs were successively distributed into the dedicated culture bag placed on the agitator contained in the device incubator. The bag was then gently agitated for 30 seconds to disperse the cell mixture, which was then incubated at 37° C. in a 5% CO₂-controlled atmosphere for a 9-day cell expansion period, without any further intervention. At the end of incubation, the cell suspension was dispersed by gentle agitation, followed by adjustment of the agitator tray to an 80° inclination to facilitate distribution of the cell suspension into two collection bags in equal volumes. Samples were collected at day 0 and day 7 to analyze sterility after dispersing the cell suspension and 50° inclination of the agitator tray.

At the end of the 9-day period, the culture product was collected, centrifuged, and immunoselected using the CliniMACS system for the purification of expanded (e) CD34+SC, which constituted the final product (ProtheraCytes®) once resuspended in 15 ml PBS/2% human serum albumin (HSA) and conditioned in three syringes of 5 ml each.

### Example 2: VEGF Quantification

The concentration of Vascular Endothelial Growth Factor (VEGF) secreted by CD34+ cells from patients with myocardial infarction (EXCELLENT Study) in cell culture supernatants was measured after 9 days of cell expansion.

In this assay, the following were tested:
16 CD34+ cell culture supernatants from AMI patients in the EXCELLENT study
4 CD34+ FHD (Frozen Healthy Donors) cell culture supernatants
3 StemFeed® media as negative controls
1 “Control Set 732 for Human VEGF” as a positive control

### Materials and Methods

Three assays were performed in triplicate according to MOP_PRD-047-Quantification of VEGF concentration using the Human VEGF QuantiGlo ELISA Kit from Bio-Techne.

TABLE 1
Assays performed

| | Assay 1 | Assay 2 | Assay 3 |
|---|---|---|---|
| VEGF concentration in | 17 May 2022 | 22 Aug. 2022 | 23 Aug. 2022 |
| supernatant_CD34+ | | | |
| cells expansion from | | | |
| AMI patients | | | |
| (EXCELLENT Study) | | | |

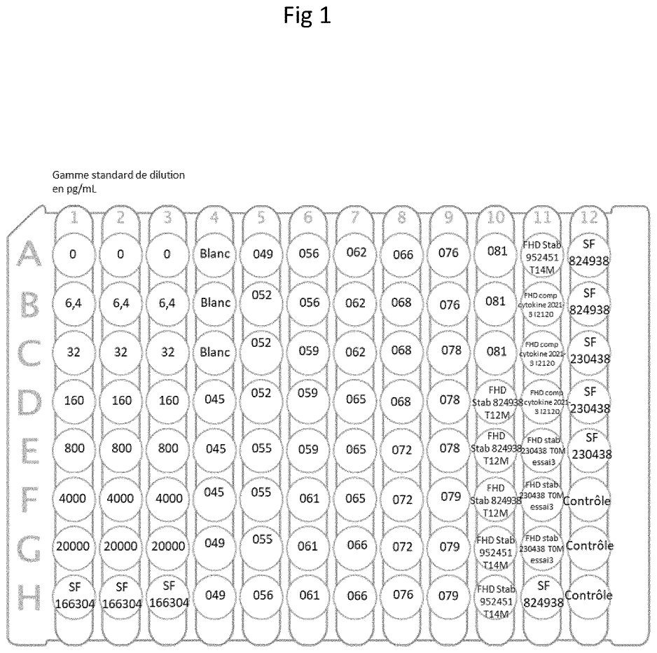

The Plate layout was as follows:
Standard range samples made from Human VEGF Standard diluted to 0; 6.4; 32; 160; 800; 4000; 20000 μg/mL,
The “0 standard” value: with the RD5L Calibration Diluent,
Blank: with the RD1-8 dilution solution of test samples,
Negative control StemFeed® samples: batches:
   166304
   824938
   230438
Patient samples: Supernatant samples collected immediately after expansion of CD34+ cells from patients 049; 052; 055; 056; 065; 066; 068; 072; 076; 078; 079 and 081,
Healthy donor samples: Supernatant samples recovered immediately after expansion of frozen healthy donor CD34+ cells (FHD_Lonza) in:
   The plate layout is shown in FIG. 1.
   StemFeed® batch 24938 stability control assay at T12M
   StemFeed® batch 952451 Stability control assay at T14M
   Cytokine mix comparison assay 3 batches K0321 vs I2120_expansion I2120
   StemFeed® stability control assay 3 batch 230438 at TOM

The plate layout is shown in FIG. 1.

### Results

### Assay 1

The tables below show the values obtained in triplicate for each sample analysed.

TABLE 2
Values obtained from the standard curve samples

| Standard | | | | | | | |
|---|---|---|---|---|---|---|---|
| range for | | | | | | | |
| standard | | | | | | | |
| curve(pg/mL) | 0 | 6.4 | 32 | 160 | 800 | 4000 | 20000 |
| Values | −5115.6 | 10638.4 | 64469.5 | 413742.5 | 2239938.3 | 9432728.3 | 44362041.2 |
| | −2285.4 | 14814.8 | 106519.5 | 489417.2 | 2844633.2 | 13984670.6 | 50507775.4 |
| | −2293.4 | 11072.1 | 95684.0 | 517820.8 | 2804636.7 | 13444521.2 | 54917368.9 |
| Mean | −3231.5 | 12175.1 | 88891.0 | 473660.2 | 2629736.1 | 12287306.7 | 49929061.8 |
| SD | 1631.7 | 2296.3 | 21832.5 | 53798.6 | 338166.6 | 2486846.1 | 5301407.2 |
| *CV % | **50.5 | 18.9 | 24.6 | 11.4 | 12.9 | 20.2 | 10.6 |

*The criterion for excluding an outlier in the calculation of the average of the values obtained for triplicates is: CV ≤ 25%.
**The CV calculated for the values obtained at point 0 of the standard curve does not take into account the exclusion criterion mentioned above due to the sensitivity of the method (sensitivity threshold of the kit ranged from 1.61-5.99 pg/mL, with a mean of 3.30 pg/mL), the values obtained being below this threshold.

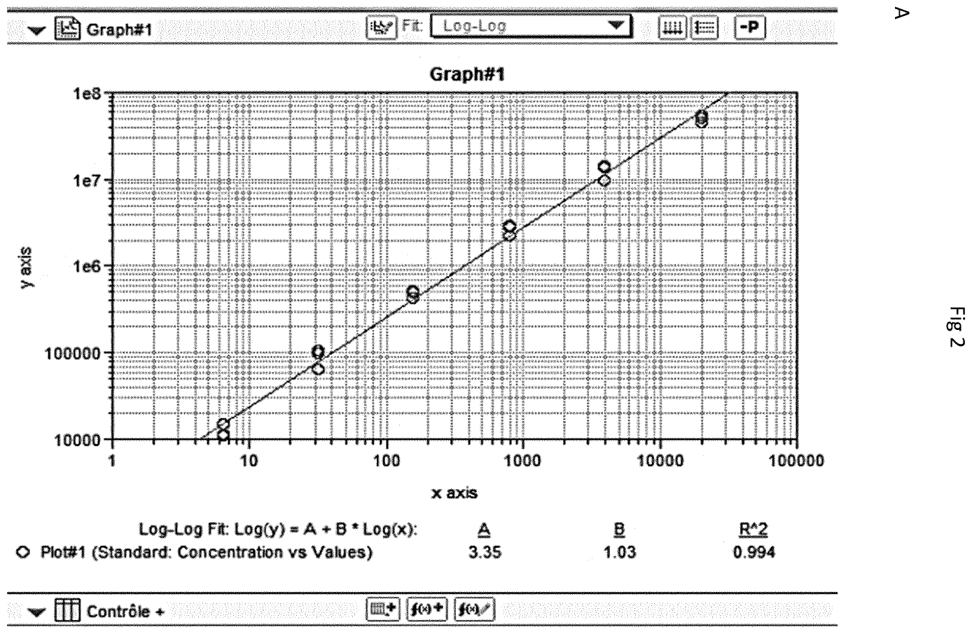

Standard curve obtained from these values is shown in FIG. 2a. The value obtained for the coefficient R\hat{ }2 (0.994) validates the standard curve obtained, the value being >0.98.

TABLE 3
Values obtained from the negative and positive controls

| | Negative | Negative | Negative | |
|---|---|---|---|---|
| | control 1 | control 2 | control 3 | Positive control |
| | StemFeed | StemFeed | StemFeed | (between 1551 and |
| Control | lot 166304 | lot 824938 | lot 230438 | 2838 pg/mL) |
| Results | Range? * | Range? | Range? | 2272.4 |
| (pg/mL) | 1.7 | Range? | Range? | 2687.4 |
| | Range? | Range? | Range? | 2506.9 |
| Mean | 1.7 | NA | NA | 2488.9 |
| SD | NA | NA | NA | 208.1 |
| CV % | NA | NA | NA | 8.4 |

* “Range?” is displayed by the luminometer when there is no detectable VEGF in the sample because of the sensitivity of the method (sensitivity threshold of the kit ranged from 1.61-5.99 pg/mL with a mean of 3.30 pg/mL). Therefore, for statistical analysis purposes, in this case this value will be arbitrarily considered as the mean of the sensitivity threshold, i.e. 3.30 pg/mL.

TABLE 4
Values obtained from the negative and positive controls
with sensitivity threshold value included

| | Negative | Negative | Negative | |
|---|---|---|---|---|
| | control 1 | control 2 | control 3 | Positive control |
| | StemFeed | StemFeed | StemFeed | (between 1551 and |
| Control | lot 166304 | lot 824938 | lot 230438 | 2838 pg/mL) |
| Results | 3.3 | 3.3 | 3.3 | 2272.4 |
| (pg/mL) | 1.7 | 3.3 | 3.3 | 2687.4 |
| | 3.3 | 3.3 | 3.3 | 2506.9 |
| Mean | NA | NA | NA | 2488.9 |
| SD | NA | NA | NA | 208.1 |
| CV % | *NA | NA | NA | 8.4 |

*NA: not applicable as the values obtained are below the sensitivity threshold of the kit 3.30 pg/mL.

TABLE 5
Values obtained from each healthy donor sample.

| | FHD_1 | FHD_2 | FHD_3 | FHD_4 |
|---|---|---|---|---|
| | Stab | Stab | Comp | Stab |
| | 824938 | 952451 | Cytokine 3 | 230438 |
| Samples | T12M | T14M | I2120 | T0M |
| Results | 351.3 | 805.3 | 412.2 | 669.5 |
| (pg/mL) | 318.1 | 738.2 | 473.2 | 753.9 |
| | 319.3 | 789.6 | 402.3 | 714.8 |
| Mean | 329.5 | 777.7 | 429.2 | 712.8 |
| SD | 18.8 | 35.1 | 38.4 | 42.2 |
| *CV % | 5.7 | 4.5 | 8.9 | 5.9 |

*The criterion for excluding an outlier in the calculation of the average of the values obtained for triplicates is: CV ≤ 25%, in this table, all CVs are below 25%, there are no outliers to exclude.

TABLE 6a
Values obtained from each patient sample

| | Patient | Patient | Patient | Patient | Patient | Patient | Patient | Patient |
|---|---|---|---|---|---|---|---|---|
| Samples | 045 | 049 | 052 | 055 | 056 | 059 | 061 | 062 |
| Results | 822.8 | 761.0 | 221.0 | 351.7 | 677.3 | 901.2 | 191.4 | 797.1 |
| (pg/mL) | 803.5 | 544.3 | 220.2 | 336.6 | 730.6 | 885.7 | 184.7 | 823.2 |
| | 1316.6* | 790.1 | 196.7 | 326.4 | 547.6 | 776.0 | 194.1 | 687.2 |
| Mean | 981.0 | 698.5 | 212.6 | 338.3 | 651.9 | 854.3 | 190.1 | 769.1 |
| SD | 290.9 | 134.3 | 13.8 | 12.7 | 94.1 | 68.2 | 4.9 | 72.2 |
| *CV % | 29.7 | 19.2 | 6.5 | 3.8 | 14.4 | 8.0 | 2.6 | 9.4 |

*The exclusion criterion for an outlier in the calculation of the mean of the triplicate values is: CV ≤ 25%, in this table only the CV of patient 045 is greater than 25%, the outlier of the triplicate is 1316.6 pg/mL and is to be excluded. Recalculation of the CV in the table below.

TABLE 6b

| | Patient | Patient | Patient | Patient | Patient | Patient | Patient | Patient |
|---|---|---|---|---|---|---|---|---|
| Sample | 045 | 049 | 052 | 055 | 056 | 059 | 061 | 062 |
| Results | 822.8 | 761.0 | 221.0 | 351.7 | 677.3 | 901.2 | 191.4 | 797.1 |
| (pg/mL) | 803.5 | 544.3 | 220.2 | 336.6 | 730.6 | 885.7 | 184.7 | 823.2 |
| | NA | 790.1 | 196.7 | 326.4 | 547.6 | 776.0 | 194.1 | 687.2 |
| Mean | 813.1 | 698.5 | 212.6 | 338.3 | 651.9 | 854.3 | 190.1 | 769.1 |
| SD | 13.6 | 134.3 | 13.8 | 12.7 | 94.1 | 68.2 | 4.9 | 72.2 |
| *CV % | 1.7 | 19.2 | 6.5 | 3.8 | 14.4 | 8.0 | 2.6 | 9.4 |
| | Patient | Patient | Patient | Patient | Patient | Patient | Patient | Patient |
| Sample | 065 | 066 | 068 | 072 | 076 | 078 | 079 | 081 |
| Results | 885.8 | 596.2 | 524.3 | 462.6 | 705.0 | 894.1 | 302.5 | 601.0 |
| (pg/mL) | 866.0 | 517.5 | 578.0 | 516.6 | 705.4 | 888.8 | 233.1 | 647.3 |
| | 921.4 | 496.5 | 469.3 | 510.7 | 597.3 | 855.7 | 224.0 | 678.7 |
| Mean | 891.1 | 536.7 | 523.9 | 496.6 | 669.2 | 879.5 | 253.2 | 642.3 |
| SD | 28.1 | 52.6 | 54.3 | 29.6 | 62.3 | 20.8 | 42.9 | 39.1 |
| *CV % | 3.1 | 9.8 | 10.4 | 6.0 | 9.3 | 2.4 | 17.0 | 6.1 |

*The criterion for excluding an outlier in the calculation of the average of the values obtained for triplicates is: CV ≤ 25%, in this table, all CVs are below 25%, there are no outliers to exclude.

### Assay 2

The tables below show the values obtained in triplicate for each sample analysed.

TABLE 7
Values obtained from the standard curve samples

| | Standard range for standard curve(pg/mL) | | | | | | |
|---|---|---|---|---|---|---|---|
| | 0 | 6.4 | 32 | 160 | 800 | 4000 | 20000 |
| Values | −319.4 | 12152.9 | 73419.8 | 345272.3 | 1934097.3 | 9749542.4 | 42224078.0 |
| | 6991.3 | 19780.1 | 75326.1 | 375548.9 | 1835640.2 | 8847823.9 | 45250516.5 |
| | 855.7 | 17158.5 | 76414.1 | 406426.2 | 2244023.1 | 12269726.9 | 50708562.3 |
| Mean | 2509.2 | 16363.8 | 75053.4 | 375749.1 | 2004586.8 | 10289031.1 | 46061052.3 |
| SD | 3925.9 | 3875.2 | 1515.7 | 30577.4 | 213121.4 | 1773595.5 | 4299923.8 |
| *CV % | **156.5 | 23.7 | 2.0 | 8.1 | 10.6 | 17.2 | 9.3 |

*The criterion for excluding an outlier in the calculation of the average of the values obtained for triplicates is: CV ≤ 25%.
**The CV calculated for the values obtained at point 0 of the standard curve does not take into account the exclusion criterion mentioned above due to the sensitivity of the method (sensitivity threshold of the kit ranged from 1.61-5.99 pg/mL with a mean of 3.30 pg/mL), the values obtained being below this threshold.

Standard curve obtained from these values is shown in FIG. 2b. The value obtained for the coefficient R\hat{ }2 (0.998) validates the standard curve obtained, the value being >0.98.

TABLE 8
Values obtained from the negative and positive controls.

| | Negative | Negative | Negative | |
|---|---|---|---|---|
| | control 1 | control 2 | control 3 | **Positive control |
| | StemFeed | StemFeed | StemFeed | (between 1551 and |
| Control | lot 166304 | lot 824938 | lot 230438 | 2838 pg/mL) |
| Results | 1.7 | * Range? | Range? | [symbol] |
| (pg/mL) | 0.8 | 0.1 | Range? | 2619.4 |
| | 5.4 | Range? | Range? | 2547.3 |
| Mean | 2.6 | 0.1 | NA | 2583.4 |
| SD | 2.4 | NA | NA | 51.0 |
| CV % | 92.1 | NA | NA | 2.0 |

* Range?” is displayed by the luminometer when there is no detectable VEGF in the sample because of the sensitivity of the method (sensitivity threshold of the kit ranged from 1.61-5.99 pg/mL with a mean of 3.30 pg/mL). Therefore, for statistical analysis purposes, in this case this value will be arbitrarily considered as the mean of the sensitivity threshold, i.e. 3.30 pg/mL.
**The value obtained in the triplicate of the positive control is out of range provided by the supplier for this control (between 1551 and 2838 pg/mL) and therefore is not retained for the calculation of the mean of this control.

TABLE 9
Values obtained from the negative and positive controls
with sensitivity threshold value included

| | Negative | Negative | Negative | |
|---|---|---|---|---|
| | control 1 | control 2 | control 3 | **Positive control |
| | StemFeed | StemFeed | StemFeed | (between 1551 and |
| Control | lot 166304 | lot 824938 | lot 230438 | 2838 pg/mL) |
| Results | 1.7 | 3.3 | 3.3 | NA |
| (pg/mL) | 0.8 | 0.1 | 3.3 | 2619.4 |
| | 5.4 | 3.3 | 3.3 | 2547.3 |
| Mean | 2.6 | 2.2 | 3.3 | 2583.4 |
| SD | 2.4 | NA | NA | 51.0 |
| CV % | NA* | NA | NA | 2.0 |

*NA: not applicable as the values obtained are below the sensitivity threshold of the kit 3.30 pg/mL.

TABLE 10
Values obtained from each healthy donor sample.

| | FHD_1 | FHD_2 | FHD_3 | FHD_4 |
|---|---|---|---|---|
| | Stab | Stab | Comp | Stab |
| | 824938 | 952451 | Cytokine 3 | 230438 |
| Samples | T12M | T14M | I2120 | T0M |
| Results | 292.4 | 681.7 | 225.8 | 568.5 |
| (pg/mL) | 268.5 | 659.1 | 295.7 | 675.6 |
| | 276.9 | 670.4 | 350.9 | 662.9 |
| Mean | 279.3 | 670.4 | 290.8 | 635.7 |
| SD | 12.1 | 11.3 | 62.7 | 58.5 |
| *CV % | 4.3 | 1.7 | 21.6 | 9.2 |

*The criterion for excluding an outlier in the calculation of the average of the values obtained for triplicates is: CV ≤ 25%, in this table, all CVs are below 25%, there are no outliers to exclude.

TABLE 11
Values obtained from each patient sample

| | Patient | Patient | Patient | Patient | Patient | Patient | Patient | Patient |
|---|---|---|---|---|---|---|---|---|
| Sample | 045 | 049 | 052 | 055 | 056 | 059 | 061 | 062 |
| Results | 1264.1 | 897.4 | 205.7 | 353.7 | 656.8 | 832.3 | 150.7 | 751.5 |
| (pg/mL) | 982.8 | 807.8 | 210.4 | 385.2 | 620.5 | 807.4 | 173.2 | 617.7 |
| | 1223.0 | 705.3 | 162.8 | 400.4 | 696.5 | 755.7 | 173.2 | 628.2 |
| Mean | 1156.6 | 803.5 | 193.0 | 379.8 | 658.0 | 798.5 | 165.7 | 665.8 |
| SD | 151.9 | 96.1 | 26.3 | 23.8 | 38.0 | 39.1 | 13.0 | 74.4 |
| *CV % | 13.1 | 12.0 | 13.6 | 6.3 | 5.8 | 4.9 | 7.8 | 11.2 |
| | Patient | | Patient | Patient | Patient | Patient | Patient | Patient |
| Sample | 065 | Patient066 | 068 | 072 | 076 | 078 | 079 | 081 |
| Results | 774.7 | 564.8 | 518.6 | 494.9 | 564.8 | 676.6 | 269.2 | 576.9 |
| (pg/mL) | 774.3 | 487.4 | 467.6 | 439.3 | 589.8 | 661.7 | 310.8 | 607.6 |
| | 843.2 | 434.8 | 495.2 | 415.9 | 616.7 | 783.9 | 251.2 | 639.2 |
| Mean | 797.4 | 495.7 | 493.8 | 450.0 | 590.4 | 707.4 | 277.1 | 607.9 |
| SD | 39.7 | 65.4 | 25.5 | 40.6 | 26.0 | 66.7 | 30.6 | 31.2 |
| *CV % | 5.0 | 13.2 | 5.2 | 9.0 | 4.4 | 9.4 | 11.0 | 5.1 |

*The criterion for excluding an outlier in the calculation of the average of the values obtained for triplicates is: CV ≤ 25%, in this tables, all CVs are below 25%, there are no outliers to exclude.

### Assay 3

The tables below show the values obtained in triplicate for each sample analysed.

TABLE 12
Values obtained from the standard curve samples

| | Standard range for standard curve(pg/mL) | | | | | | |
|---|---|---|---|---|---|---|---|
| | 0 | 6.4 | 32 | 160 | 800 | 4000 | 20000 |
| Values | 6592.7 | 5462.2 | 70417.3 | 395696.0 | 1999198.4 | 11693375.2 | 42014887.3 |
| | −330.6 | 10811.9 | 63601.2 | 357218.3 | 1934369.9 | 10899245.3 | 44282831.8 |
| | −6230.0 | 9478.8 | 93590.8 | 405127.9 | 2173180.9 | 13455525.8 | 47715340.0 |
| Mean | 10.7 | 8584.3 | 75869.8 | 386014.1 | 2035583.1 | 12016048.8 | 44671019.7 |
| SD | 6418.1 | 2784.8 | 15720.7 | 25379.9 | 123493.1 | 1308331.4 | 2869983.9 |
| *CV % | **59945.2 | ***32.4 | 20.7 | 6.6 | 6.1 | 10.9 | 6.4 |

*The criterion for excluding an outlier in the calculation of the average of the values obtained for triplicates is: CV ≤ 25%.
**The CV calculated for the values obtained at point 0 of the standard curve does not take into account the exclusion criterion mentioned above due to the sensitivity of the method (sensitivity threshold of the kit ranged from 1.61-5.99 pg/mL with a mean of 3.30 pg/mL), the values obtained being below this threshold.
***Only the CV for 6.4 is greater than 25%, the triplicate outlier is 5462.2 pg/mL and should be excluded.

Recalculation of the CV in Table 13 below.

TABLE 13
Recalculated Values obtained from the standard curve samples

| | Standard range for standard curve(pg/mL) | | | | | | |
|---|---|---|---|---|---|---|---|
| | 0 | 6.4 | 32 | 160 | 800 | 4000 | 20000 |
| Values | 6592.7 | NA | 70417.3 | 395696.0 | 1999198.4 | 11693375.2 | 42014887.3 |
| | −330.6 | 10811.9 | 63601.2 | 357218.3 | 1934369.9 | 10899245.3 | 44282831.8 |
| | −6230.0 | 9478.8 | 93590.8 | 405127.9 | 2173180.9 | 13455525.8 | 47715340.0 |
| Mean | 10.7 | 10145.3 | 75869.8 | 386014.1 | 2035583.1 | 12016048.8 | 44671019.7 |
| SD | 6418.1 | 942.6 | 15720.7 | 25379.9 | 123493.1 | 1308331.4 | 2869983.9 |
| *CV % | *NA | 9.3 | 20.7 | 6.6 | 6.1 | 10.9 | 6.4 |

*NA: not applicable as the values obtained are below the sensitivity threshold of the kit 3.30 pg/mL.

Standard curve obtained from these values is shown in FIG. 2c. The value obtained for the coefficient R\hat{ }2 (0.996) validates the standard curve obtained, the value being >0.98.

TABLE 14
Values obtained from the negative and positive controls

| | Negative | Negative | Negative | Positive control |
|---|---|---|---|---|
| | control 1 | control 2 | control 3 | (between 1551 |
| | StemFeed | StemFeed | StemFeed | and 2838 |
| Control | lot 166304 | Lot 824938 | lot 230438 | pg/mL) |
| Results | 2.0 | *Range? | 3.2 | 2628.6 |
| (pg/mL) | 0.9 | 2.8 | 1.2 | 2477.1 |
| | 4.7 | 4.8 | 0.8 | 2565.6 |
| Mean | 2.5 | 3.8 | 1.7 | 2557.1 |
| SD | 1.9 | 1.4 | 1.2 | 76.1 |
| CV % | 76.6 | 38.0 | 71.7 | 3.0 |

*“Range?” is displayed by the luminometer when there is no detectable VEGF in the sample because of the sensitivity of the method (sensitivity threshold of the kit ranged from 1.61-5.99 pg/mL with a mean of 3.30 pg/mL). Therefore, for statistical analysis purposes, in this case this value will be arbitrarily considered as the mean of the sensitivity threshold, i.e. 3.30 pg/mL.
**The value obtained in the triplicate of the positive control is out of range provided by the supplier for this control (between 1551 and 2838 pg/mL) and therefore is not retained for the calculation of the mean of this control.

TABLE 15
Values obtained from the negative and positive controls
with sensitivity threshold value included

| | Negative | Negative | Negative | |
|---|---|---|---|---|
| | control 1 | control 2 | control 3 | Positive control |
| | StemFeed | StemFeed | StemFeed | (between 1551 and |
| Control | lot 166304 | L*ot 824938 | lot 230438 | 2838 pg/mL) |
| Results | 2.0 | 3.3 | 3.2 | 2628.6 |
| (pg/mL) | 0.9 | 2.8 | 1.2 | 2477.1 |
| | 4.7 | 4.8 | 0.8 | 2565.6 |
| Mean | 2.5 | 3.6 | 1.7 | 2557.1 |
| SD | 1.9 | 1.1 | 1.2 | 76.1 |
| CV % | NA | NA | NA | 3.0 |

*NA: not applicable as the values obtained are below the sensitivity threshold of the kit 3.30 pg/mL.

TABLE 16
Values obtained from each healthy donor sample.

| | FHD_1 | FHD_2 | FHD_3 | FHD_4 |
|---|---|---|---|---|
| | Stab | Stab | Comp | Stab |
| | 824938 | 952451 | Cytokine 3 | 230438 |
| Samples | T12M | T14M | I2120 | T0M |
| Results | 373.4 | 658.0 | 305.9 | 605.2 |
| (pg/mL) | 293.5 | 739.2 | 390.9 | 686.8 |
| | 344.4 | 723.6 | 380.0 | 707.5 |
| Mean | 337.1 | 706.9 | 359.0 | 666.5 |
| SD | 40.5 | 43.1 | 46.2 | 54.1 |
| CV % | 12.0 | 6.1 | 12.9 | 8.1 |

*The criterion for excluding an outlier in the calculation of the average of the values obtained for triplicates is: CV ≤ 25%, in this table, all CVs are below 25%, there are no outliers to exclude.

TABLE 17
Values obtained from each patient sample

| | Patient | Patient | Patient | Patient | Patient | Patient | Patient | Patient |
|---|---|---|---|---|---|---|---|---|
| Sample | 045 | 049 | 052 | 055 | 056 | 059 | 061 | 062 |
| Results | 1110.7 | 830.5 | 221.9 | 377.9 | 697.7 | 948.9 | 209.4 | 708.8 |
| (pg/mL) | 1071.9 | 727.2 | 222.5 | 435.2 | 657.7 | 892.9 | 194.5 | 614.2 |
| | 1198.3 | 753.3 | 176.4 | 448.5 | 726.9 | 802.2 | 199.5 | 766.8 |
| Mean | 1127.0 | 770.3 | 206.9 | 420.5 | 694.1 | 881.3 | 201.1 | 696.6 |
| SD | 64.8 | 53.7 | 26.4 | 37.5 | 34.8 | 74.0 | 7.6 | 77.0 |
| CV % | 5.7 | 7.0 | 12.8 | 8.9 | 5.0 | 8.4 | 3.8 | 11.1 |
| | Patient | Patient | Patient | Patient | Patient | Patient | Patient | Patient |
| Sample | 065 | 066 | 068 | 072 | 076 | 078 | 079 | 081 |
| Results | 680.2 | 588.3 | 538.6 | 405.2 | 632.6 | 827.9 | 449.4 | 576.4 |
| (pg/mL) | 931.5 | 611.7 | 589.0 | 440.9 | 580.8 | 847.2 | 412.0 | 634.3 |
| | 1003.0 | 581.3 | 436.3 | 509.5 | 653.9 | 829.8 | 432.2 | 686.7 |
| Mean | 871.6 | 593.8 | 521.3 | 451.9 | 622.4 | 834.9 | 431.2 | 632.4 |
| SD | 169.5 | 15.9 | 77.8 | 53.0 | 37.6 | 10.6 | 18.7 | 55.2 |
| CV % | 19.4 | 2.7 | 14.9 | 11.7 | 6.0 | 1.3 | 4.3 | 8.7 |

* The criterion for excluding an outlier in the calculation of the average of the values obtained for triplicates is: CV ≤ 25%, in this table, all CVs are below 25%, there are no outliers to exclude.

### Analysis and Observations

### Assay 1

### VEGF Concentration

The averages of triplicate measurements for VEGF concentration in 9-day culture supernatants from frozen healthy donor CD34+ cells (FHD) and from CD34+ cells from patients with myocardial infarction are given in the table below.

TABLE 18
VEGF concentration in 9-day culture supernatants from patient CD34+ cells

| | Patient | Patient | Patient | Patient | Patient | Patient | Patient | Patient |
|---|---|---|---|---|---|---|---|---|
| Samples | 045 | 049 | 052 | 055 | 056 | 059 | 061 | 062 |
| Mean (pg/mL) | 813.2 | 698.5 | 212.6 | 338.2 | 651.8 | 854.3 | 190.1 | 769.2 |
| SD | 13.7 | 134.3 | 13.8 | 12.7 | 94.1 | 68.3 | 4.8 | 72.2 |
| | Patient | Patient | Patient | Patient | Patient | Patient | Patient | Patient |
| Samples | 065 | 066 | 068 | 072 | 076 | 078 | 079 | 081 |
| Mean (pg/mL) | 891.1 | 536.7 | 523.9 | 496.6 | 669.2 | 879.5 | 253.2 | 642.3 |
| SD | 28.1 | 52.6 | 54.4 | 29.6 | 62.3 | 20.8 | 42.9 | 39.1 |

TABLE 19
VEGF concentration in 9-day culture supernatants from healthy donor CD34+ cells and
controls

| | | | | | | | | Positive |
|---|---|---|---|---|---|---|---|---|
| | | | | | | | | control |
| | FHD_1 | FHD_2 | FHD_3 | FHD_4 | Negative | Negative | Negative | (between |
| | Stab | Stab | Comp | Stab | control | control | control | 1551 and |
| | 824938 | 952451 | Cytokine | 230438 | StemFeed ® | StemFeed ® | StemFeed ® | 2838 |
| Samples | T12M | T14M | 3 I2120 | T0M | lot 166304 | lot 824938 | lot 230438 | pg/mL) |
| Mean | 329.6 | 777.7 | 429.2 | 712.7 | 2.8 | 3.3 | 3.3 | 2488.9 |
| (pg/mL) | | | | | | | | |
| SD | 18.8 | 35.1 | 38.4 | 42.2 | 0.9 | 0.0 | 0.0 | 208.1 |

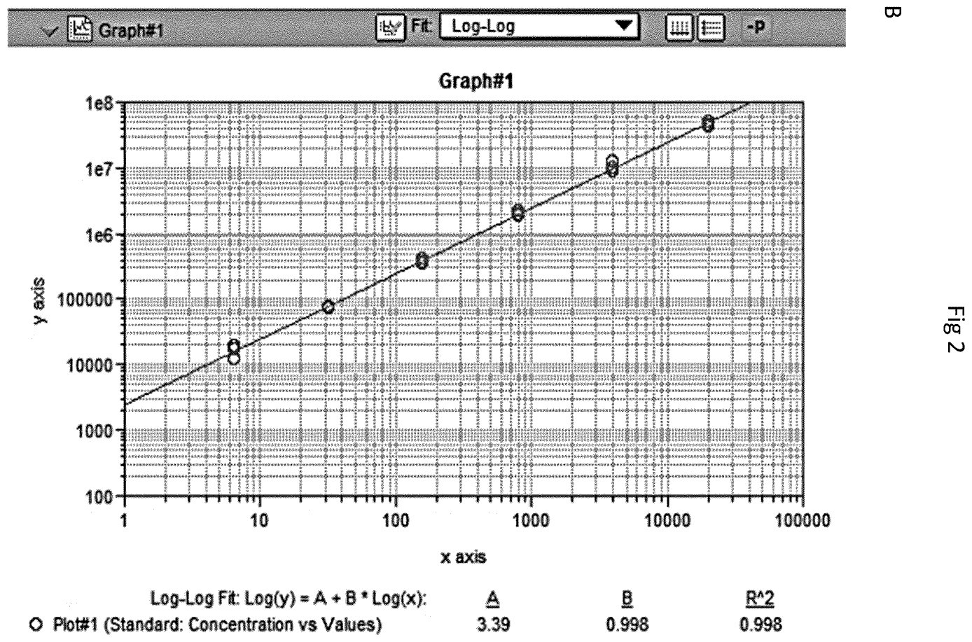

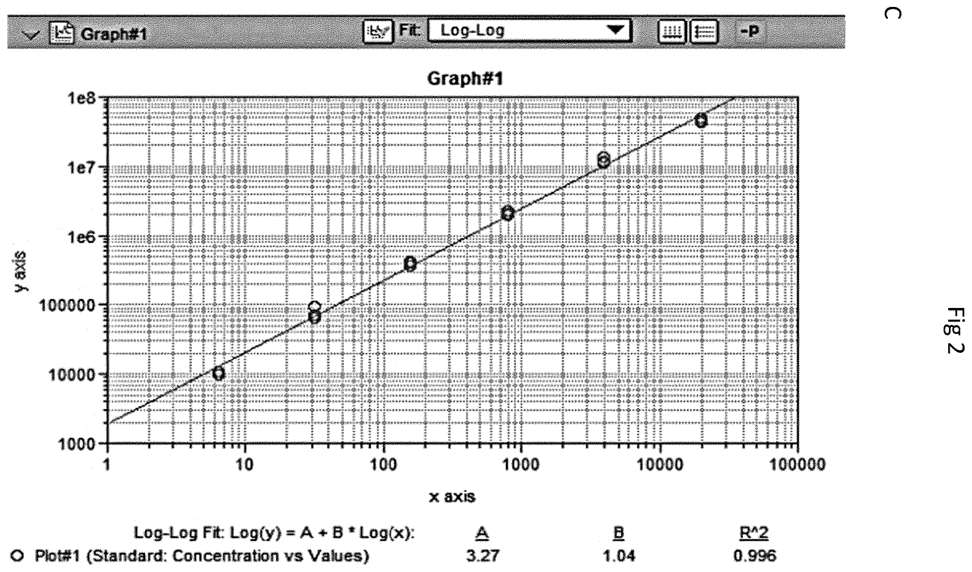

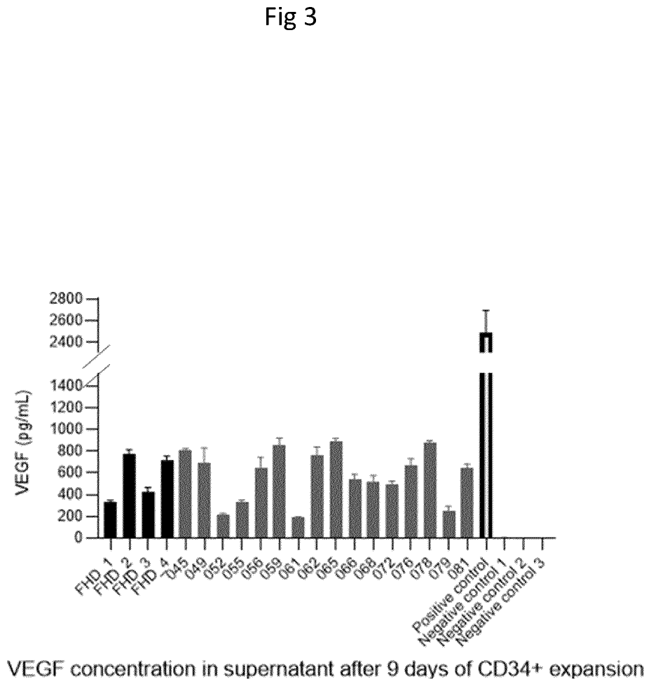

These results are also shown in FIG. 3.
The VEGF concentration measured for the positive control (Human VEGF) is 2488.9±208.1 pg/mL. This concentration is within the specification provided by Bio&Techne for this control. This value should be between 1551 and 2838 μg/mL.
VEGF concentration measured for the negative control (StemFeed® medium alone) is:
   StemFeed® lot 166304: 2.8±0.9 μg/mL,
   StemFeed® lot 824938: 3.3±0.0 μg/mL,
   StemFeed® lot 230438: 3.3±0.0 μg/mL.
VEGF concentration measured in the supernatant after 9 days of expansion of patient CD34+ cells ranged from 190.1±4.8 μg/mL (patient 061) to 891.1±28.1 μg/mL (patient 065).
The VEGF concentration measured in the supernatant after 9 days of expansion of healthy donor CD34± cells ranged from 329.6±18.8 μg/mL (FHD_1) to 777.7±35.1 μg/mL (FHD_2).

The average VEGF concentrations obtained from CD34+ cell expansions of 4 healthy donors, 16 patients, and 3 culture media alone (StemFeed® without cytokines) as negative controls are shown in Table 20 below.

TABLE 20
average VEGF concentrations obtained from CD34+ cell
expansions of 4 healthy donors, 16 patients, and 3 negative
controls (culture media alone StemFeed ® without cytokines)

| | | | Negative |
|---|---|---|---|
| | | | control |
| Samples | FHD | Patients | StemFeed ® |
| Minimum value | 329.6 | 190.1 | 2.8 |
| Maximum value | 777.7 | 891.1 | 3.3 |
| Mean (pg/mL) | 562.3 | 588.8 | 3.1 |
| SD | 216.7 | 237.5 | 0.3 |

The mean value of VEGF concentration obtained from 4 healthy donors (FHD) is 562.3±216.7 pg/mL with a minimum value of 329.6 μg/mL (FHD_1) and a maximum value of 777.7 μg/mL (FHD_2).
The mean value of VEGF concentration obtained from 16 patients is 588.8±237.5 μg/mL with a minimum value of 190.1 μg/mL (patient 052) and maximum of 891.1 μg/mL (patient 065).
The mean value of VEGF concentration obtained from 3 StemFeed® culture media (negative controls) is 3.1±0.3 μg/mL with values: minimum of 2.8 μg/mL (StemFeed® lot 166304) and maximum of 3.3 μg/mL (StemFeed® lot 824938 and lot 230438), values assigned when the luminometer displays “Range?”.

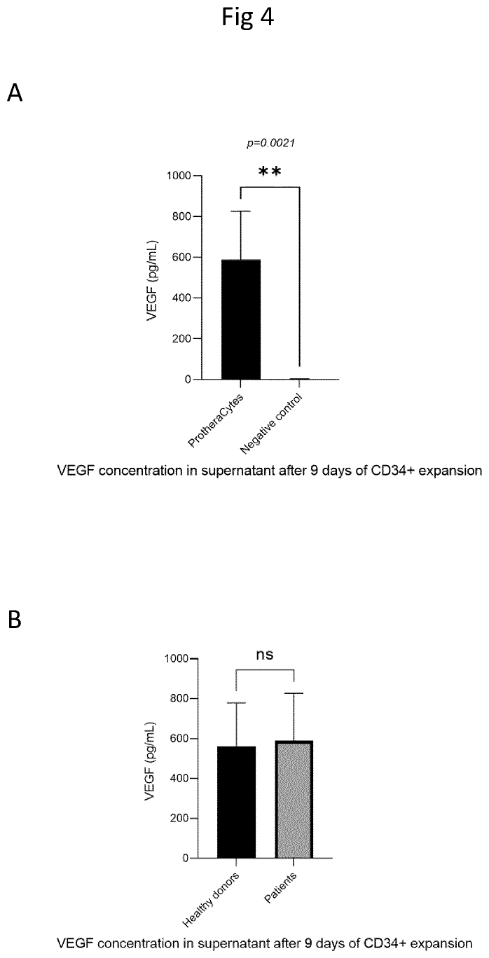

Finally, there is a significant difference between the VEGF concentration in patient supernatant and the VEGF concentration in StemFeed® culture medium alone (Mann Whitney test, p=0.0021; FIG. 4a), but no significant difference was shown when the VEGF concentration in patient supernatant was compared to the VEGF concentration in healthy donor supernatant (t-test, p=0.8420; FIG. 4b).

Correlation Between VEGF Concentration and Number of CD34+ Cells Obtained after Expansion

Table 21 below shows data from ProtheraCytes® manufacturing from patients in the EXCELLENT study.

TABLE 21
Number of CD34+ cells after 9 days of expansion

| ID | Number of CD34+ cells |
|---|---|
| Patient | after culture (×10⁶) |
| 045 | 182.7 |
| 049 | 44.3 |
| 052 | 36.7 |
| 055 | 64.6 |
| 056 | 41.9 |
| 059 | 256.4 |
| 061 | 29.8 |
| 062 | 165.1 |
| 065 | 211.6 |
| 066 | 73.1 |
| 068 | 62.6 |
| 072 | 75.0 |
| 076 | 79.0 |
| 078 | 226.9 |
| 079 | 58.7 |
| 081 | 101.9 |

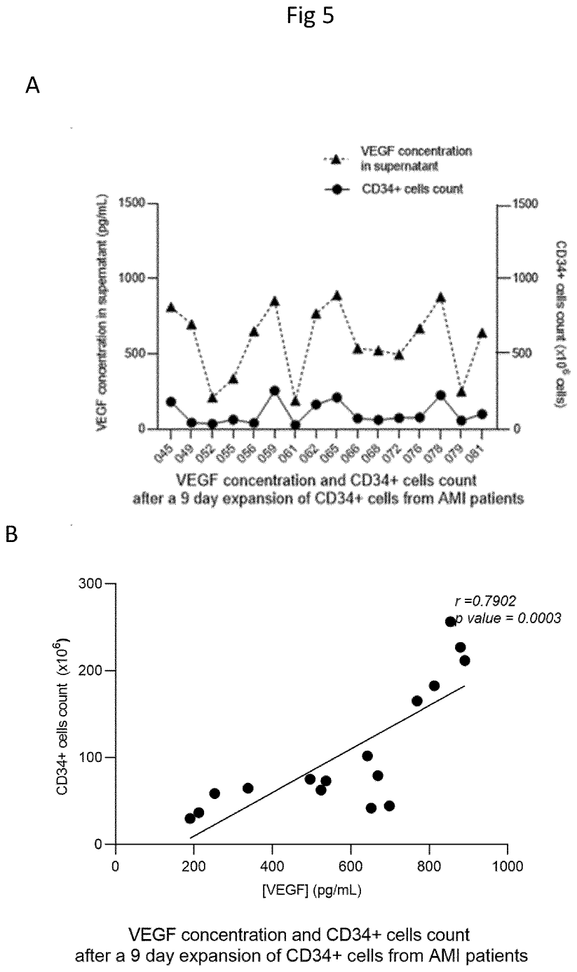

These results are also shown in FIG. 5.

FIG. 5A demonstrates that the curve obtained on the concentration of VEGF in the supernatant mimics the curve from the number of CD34+ cells obtained after 9 days of cell expansion. FIG. 5B shows that there is a positive significant correlation between VEGF concentration and number of CD34+ cells after expansion (Pearson correlation coefficient=0,7902; p value=0,0003).

### Assay 2

### VEGF Concentration

The average of triplicate measurements for VEGF concentration in 9-day culture supernatants from CD34+ cells from patients with myocardial infarction and from frozen healthy donor CD34+ cells (FHD) were measured and are shown in Tables 22 and 23 below.

TABLE 22
VEGF concentration in 9-day culture supernatants from myocardial
infarction patient CD34+ cells

| | Patient | Patient | Patient | Patient | Patient | Patient | Patient | Patient |
|---|---|---|---|---|---|---|---|---|
| Samples | 045 | 049 | 052 | 055 | 056 | 059 | 061 | 062 |
| Mean | 1156.6 | 803.5 | 193.0 | 379.8 | 657.9 | 798.5 | 165.7 | 665.8 |
| (pg/mL) | | | | | | | | |
| SD | 151.9 | 96.1 | 26.2 | 23.8 | 38.0 | 39.1 | 13.0 | 74.4 |
| | Patient | Patient | Patient | Patient | Patient | Patient | Patient | Patient |
| Samples | 065 | 066 | 068 | 072 | 076 | 078 | 079 | 081 |
| Mean | 797.4 | 495.7 | 493.8 | 450.0 | 590.4 | 707.4 | 277.1 | 607.9 |
| (pg/mL) | | | | | | | | |
| SD | 39.7 | 65.4 | 25.5 | 40.6 | 26.0 | 66.7 | 30.6 | 31.2 |

TABLE 23
VEGF concentration in 9-day culture supernatants from FHD CD34+ cells

| | | | | | | | | Positive |
|---|---|---|---|---|---|---|---|---|
| | | | | | | | | control |
| | FHD_1 | FHD_2 | FHD_3 | FHD_4 | Negative | Negative | Negative | (between |
| | Stab | Stab | Comp | Stab | control | control | control | 1551 and |
| | 824938 | 952451 | Cytokine | 230438 | StemFeed ® | StemFeed ® | StemFeed ® | 2838 |
| Samples | T12M | T14M | 3 I2120 | T0M | lot 166304 | lot 824938 | lot 230438 | pg/mL) |
| Mean | 279.3 | 670.4 | 290.8 | 635.7 | 2.6 | 2.2 | 3.3 | 2583.4 |
| (pg/mL) | | | | | | | | |
| SD | 12.1 | 11.3 | 62.7 | 58.5 | 2.4 | 1.8 | 0.0 | 51.0 |

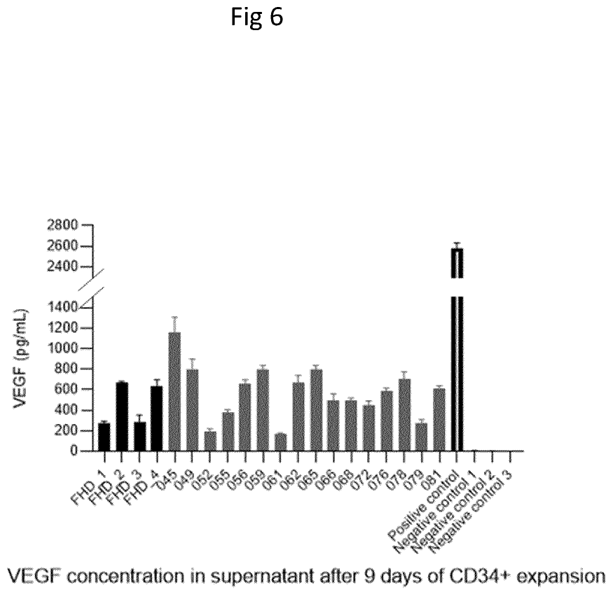

These results are also shown in FIG. 6.
The VEGF concentration measured for the positive control (Human VEGF) is 2583.4±51.0 μg/mL. This concentration is within the specification provided by Bio&Techne for this control. This value should be between 1551 and 2838 μg/mL.
VEGF concentration measured for the negative control (StemFeed® medium alone) is:
   StemFeed® lot 166304:2.6±2.4 μg/mL,
   StemFeed® lot 824938:2.2±1.8 μg/mL,
   StemFeed® lot 230438:3.3±0.0 μg/mL.
VEGF concentration measured in the supernatant after 9 days of expansion of patient CD34+ cells ranged from 165.7±13.0 μg/mL (patient 061) to 1156.6±151.9 μg/mL (patient 045).
The VEGF concentration measured in the supernatant after 9 days of expansion of healthy donor CD34± cells ranged from 279.3±12.1 μg/mL (FHD_1) to 670.4±11.3 μg/mL (FHD_2).

The average VEGF concentrations obtained from CD34+ cell expansions of 4 healthy donors, 16 patients, and 3 culture media alone (StemFeed® without cytokines) as negative controls are shown in Table 24 below.

TABLE 24
VEGF concentrations obtained from CD34+ cell expansions
of heatlhy donors and patients, and negative controls

| | | | Negative |
|---|---|---|---|
| | | | control |
| Samples | FHD | Patients | StemFeed ® |
| Minimum value | 279.3 | 165.7 | 2.2 |
| Maximum value | 670.4 | 1156.6 | 3.3 |
| Mean (pg/mL) | 469.1 | 577.6 | 2.7 |
| SD | 213.0 | 257.3 | 0.5 |

The mean value of VEGF concentration obtained from 4 healthy donors (FHD) is 469.1±213.0 μg/mL with a minimum value of 279.3 μg/mL pg/mL (FHD_1) and a maximum value of 670.4 μg/mL (FHD_2).
The mean value of VEGF concentration obtained from 16 patients is 577.6±257.3 μg/mL with a minimum value of 165.7 μg/mL (patient 061) and maximum of 1156.6 μg/mL (patient 045).
The mean value of VEGF concentration obtained from 3 StemFeed® culture media (negative control) is 2.7±0.5 μg/mL with a minimum value of 2.2 μg/mL (StemFeed® lot 824938) and a maximum of 330 μg/mL (StemFeed® lot 166304, value assigned when the luminometer displays “Range?”).

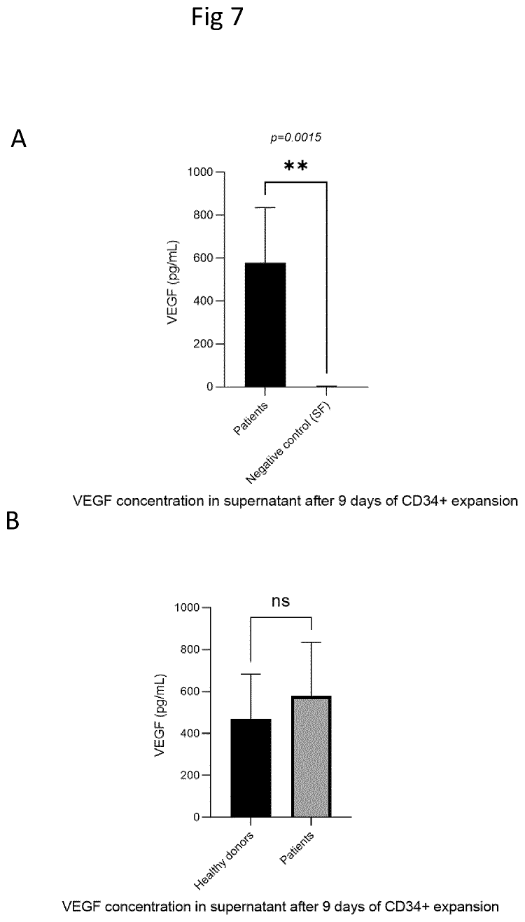

Finally, there is a significant difference between the VEGF concentration in patient supernatant and the VEGF concentration in StemFeed® culture medium alone (t-test, p=0.0015; see FIG. 7A), but no significant difference was shown when the VEGF concentration in patient supernatant was compared to the VEGF concentration in healthy donor supernatant (t-test, p=0.4484; see FIG. 7B).

Correlation Between VEGF Concentration and Number of CD34+ Cells Obtained after Expansion

Table 25 below shows data from ProtheraCytes® manufacturing from patients in the EXCELLENT study.

TABLE 25
Number of CD34+ cells after 9 days of expansion

| | Number of CD34+ cells |
|---|---|
| ID Patient | after culture (×10⁶) |
| 045 | 182.7 |
| 049 | 44.3 |
| 052 | 36.7 |
| 055 | 64.6 |
| 056 | 41.9 |
| 059 | 256.4 |
| 061 | 29.8 |
| 062 | 165.1 |
| 065 | 211.6 |
| 066 | 73.1 |
| 068 | 62.6 |
| 072 | 75.0 |
| 076 | 79.0 |
| 078 | 226.9 |
| 079 | 58.7 |
| 081 | 101.9 |

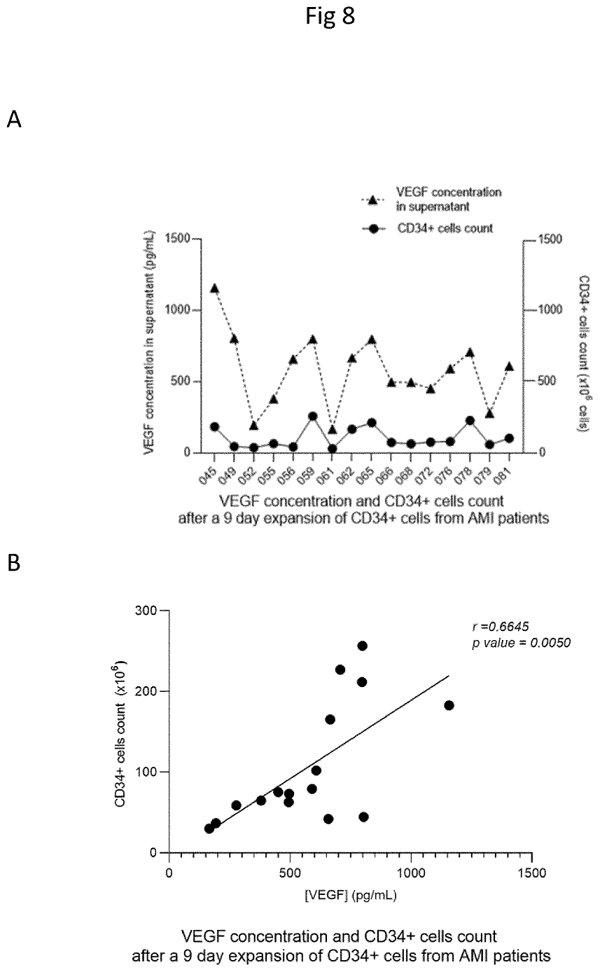

These results are also shown in FIG. 8.

FIG. 8A demonstrates that the curve obtained on the concentration of VEGF in the supernatant mimics the curve from the number of CD34+ cells obtained after 9 days of cell expansion. FIG. 8B shows that there is a positive significant correlation between VEGF concentration and number of CD34+ cells after expansion (Pearson correlation coefficient r=0.6645; p value=0.0050).

### Assay 3

### VEGF Concentration

The averages of triplicate measurements for VEGF concentration in 9-day culture supernatants from patients with myocardial infarction and from frozen healthy donor CD34+ cells (FHD) and from CD34+ cells were measured and are shown in Tables 26 and 27 below.

TABLE 26
VEGF concentration in 9-day culture supernatants from myocardial infarction
patient CD34+ cells

| | Patient | Patient | Patient | Patient | Patient | Patient | Patient | Patient |
|---|---|---|---|---|---|---|---|---|
| Samples | 045 | 049 | 052 | 055 | 056 | 059 | 061 | 062 |
| Mean (pg/mL) | 1127.0 | 770.3 | 206.9 | 420.5 | 694.1 | 881.3 | 201.1 | 696.6 |
| SD | 64.8 | 53.7 | 26.4 | 37.5 | 34.7 | 74.0 | 7.6 | 77.0 |
| | Patient | Patient | Patient | Patient | Patient | Patient | Patient | Patient |
| Samples | 065 | 066 | 068 | 072 | 076 | 078 | 079 | 081 |
| Mean (pg/mL) | 871.6 | 593.8 | 521.3 | 451.9 | 622.4 | 835.0 | 431.2 | 632.5 |
| SD | 169.5 | 15.9 | 77.8 | 53.0 | 37.6 | 10.6 | 18.7 | 55.2 |

TABLE 27
VEGF concentration in 9-day culture supernatants from FHD CD34+ cells

| | | | | | | | | Positive |
|---|---|---|---|---|---|---|---|---|
| | | | | | | | | control |
| | FHD_1 | FHD_2 | FHD_3 | FHD_4 | Negative | Negative | Negative | (between |
| | Stab | Stab | Comp | Stab | control | control | control | 1551 and |
| | 824938 | 952451 | Cytokine | 230438 | StemFeed ® | StemFeed ® | StemFeed ® | 2838 |
| Samples | T12M | T14M | 3 I2120 | T0M | lot 166304 | lot 824938 | lot 230438 | pg/mL) |
| Mean | 337.1 | 706.9 | 358.9 | 666.5 | 2.5 | 3.6 | 1.7 | 2557.1 |
| (pg/mL) | | | | | | | | |
| SD | 40.5 | 43.1 | 46.3 | 54.1 | 2.0 | 1.0 | 1.3 | 76.1 |

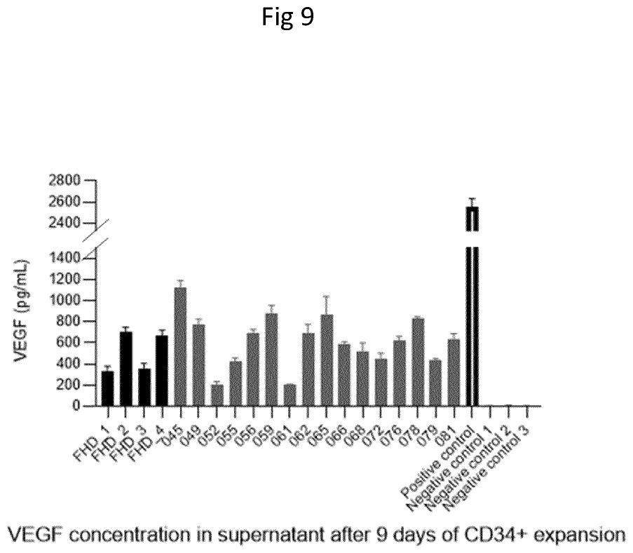

These results are also shown in FIG. 9.
VEGF concentration measured for the positive control (Human VEGF) is 2557.1±76.1 μg/mL. This concentration is within the specification provided by Bio&Techne for this control. This value should be between 1551 and 2838 μg/mL.
VEGF concentration measured for the negative control (StemFeed® medium alone) is:
   StemFeed® lot 166304:2.5±2.0 μg/mL,
   StemFeed® lot 824938:3.6±1.0 μg/mL,
   StemFeed® lot 230438:1.7±1.3 μg/mL.
VEGF concentration measured in the supernatant after 9 days of expansion of patient CD34+ cells ranged from 201.1±7.6 μg/mL (patient 061) to 1127.0±64.8 μg/mL (patient 045).
The VEGF concentration measured in the supernatant after 9 days of expansion of healthy donor CD34± cells ranged from 337.1±40.5 μg/mL (FHD_1) to 706.9±43.1 μg/mL (FHD_2).

The average VEGF concentrations obtained from CD34+ cell expansions of 4 healthy donors, 16 patients, and 3 culture media alone (StemFeed® without cytokines) as negative controls are shown in Table 28 below.

TABLE 28
VEGF concentrations obtained from CD34+ cell expansions
of heatlhy donors and patients, and negative controls

| | | | Negative control |
|---|---|---|---|
| Samples | FHD | Patients | StemFeed ® |
| Minimum value | 337.1 | 201.1 | 1.7 |
| Maximum value | 706.9 | 1127.0 | 3.6 |
| Mean (pg/mL) | 517.4 | 622.3 | 2.6 |
| SD | 196.4 | 248.9 | 1.0 |

The mean value of VEGF concentration obtained from 4 healthy donors (FHD) is 517.4±196.4 μg/mL with a minimum value of 337.1 μg/mL (FHD_1) and a maximum value of 706.9 μg/mL (FHD_2).
The mean value of VEGF concentration obtained from 16 patients is 622.3±248.9 μg/mL with a minimum value of 201.1 μg/mL (patient 061) and maximum of 1127.0 μg/mL (patient 045).
The mean value of VEGF concentration obtained from 3 StemFeed® is 2.6±1.0 μg/mL with a minimum value of 1.7 μg/mL (StemFeed® lot 230438) and a maximum of 3.6 μg/mL (StemFeed® lot 824938).

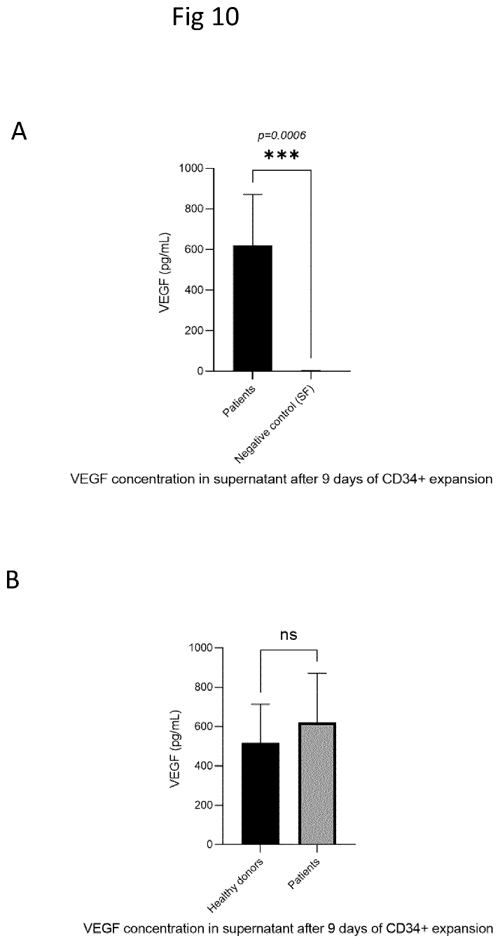

Finally, there is a significant difference between the VEGF concentration in patient supernatant and the VEGF concentration in StemFeed® culture medium alone (t-test, p=0.0006; see FIG. 10A), but no significant difference was shown when the VEGF concentration in patient supernatant was compared to the VEGF concentration in healthy donor supernatant (t-test, p=0.4458; see FIG. 10B).

Correlation Between VEGF Concentration and Number of CD34+ Cells Obtained after Expansion

Table 29 below shows data from ProtheraCytes® manufacturing from patients in the EXCELLENT study.

TABLE 29
Number of CD34+ cells after 9 days of expansion

| | Number of CD34+ cells |
|---|---|
| ID Patient | after culture (×10⁶) |
| 045 | 182.7 |
| 049 | 44.3 |
| 052 | 36.7 |
| 055 | 64.6 |
| 056 | 41.9 |
| 059 | 256.4 |
| 061 | 29.8 |
| 062 | 165.1 |
| 065 | 211.6 |
| 066 | 73.1 |
| 068 | 62.6 |
| 072 | 75.0 |
| 076 | 79.0 |
| 078 | 226.9 |
| 079 | 58.7 |
| 081 | 101.9 |

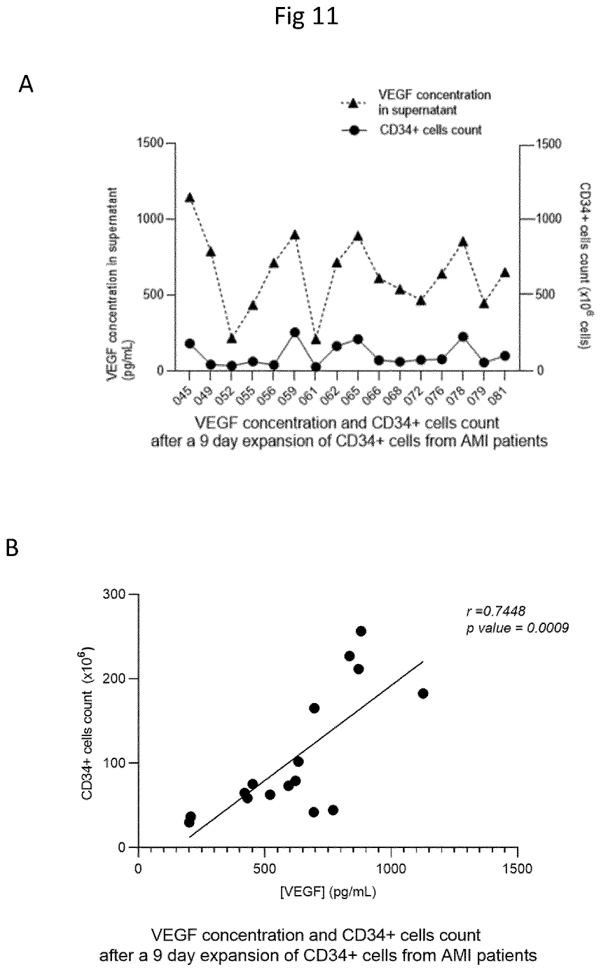

This data is also shown in FIG. 11.

FIG. 11A demonstrates that the curve obtained on the concentration of VEGF in the supernatant mimics the curve from the number of CD34+ cells obtained after 9 days of cell expansion. FIG. 11B shows that there is a positive significant correlation between VEGF concentration and number of CD34+ cells after expansion (Pearson correlation coefficient r=0.7448; p value=0.0009).

### Results of the Average of the 3 Assays (1, 2 and 3)

### VEGF Concentration

TABLE 30
VEGF concentration in 9-day culture supernatants from myocardial
infarction patient CD34+ cells

| | Patient | Patient | Patient | Patient | Patient | Patient | Patient | Patient |
|---|---|---|---|---|---|---|---|---|
| Samples | 045 | 049 | 052 | 055 | 056 | 059 | 061 | 062 |
| Mean (pg/mL) | 1032.4 | 757.4 | 204.2 | 379.5 | 667.9 | 844.7 | 185.6 | 710.5 |
| SD | 190.4 | 53.7 | 10.1 | 41.2 | 22.9 | 42.2 | 18.1 | 53.1 |
| CV % | 18.4 | 7.1 | 4.9 | 10.8 | 3.4 | 5.0 | 9.8 | 7.5 |
| | Patient | Patient | Patient | Patient | Patient | Patient | Patient | Patient |
| Samples | 065 | 066 | 068 | 072 | 076 | 078 | 079 | 081 |
| Mean (pg/mL) | 853.4 | 542.1 | 513.0 | 466.2 | 627.3 | 807.3 | 320.5 | 627.6 |
| SD | 49.4 | 49.3 | 16.7 | 26.4 | 39.6 | 89.3 | 96.6 | 17.7 |
| CV % | 5.8 | 9.1 | 3.3 | 5.7 | 6.3 | 11.1 | 30.1 | 2.8 |

TABLE 31
VEGF concentration in 9-day culture supernatants from FHD CD34+ cells

| | | | | | | | | Positive |
|---|---|---|---|---|---|---|---|---|
| | | | | | | | | control |
| | | | | | | | | (between |
| | FHD_1 | FHD_2 | FHD_3 | FHD 4 | Negative | Negative | Negative | 1551 |
| | Stab | Stab | Comp | Stab | control | control | control | and |
| | 824938 | 952451 | Cytokine | 230438 | StemFeed ® | StemFeed ® | StemFeed ® | 2838 |
| Samples | T12M | T14M | 3 I2120 | T0M | lot 166304 | lot 824938 | lot 230438 | pg/mL) |
| Mean | 315.3 | 718.3 | 359.6 | 671.6 | 2.6 | 3.1 | 2.8 | 2543.0 |
| (pg/mL) | | | | | | | | |
| SD | 31.4 | 54.6 | 69.2 | 38.8 | 0.1 | 0.7 | 0.9 | 48.5 |
| CV % | 10.0 | 7.6 | 19.2 | 5.8 | 4.4 | 23.9 | 32.6 | 1.9 |

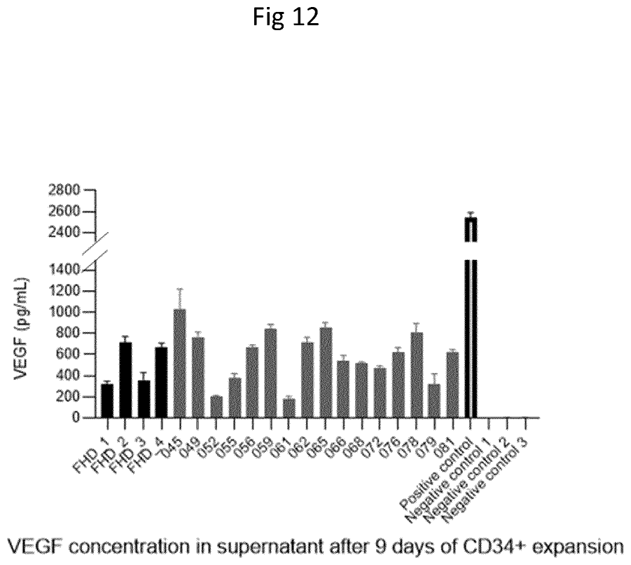

These results are also shown in FIG. 12.
The VEGF concentration measured for the positive control (Human VEGF) is 2543.0±48.5 μg/mL. This concentration is within the specification provided by Bio&Techne for this control. This value should be between 1551 and 2838 μg/mL.
VEGF concentration measured for the negative control (StemFeed® medium alone) is:
   StemFeed® lot 166304:2.6±0.1 μg/mL,
   StemFeed® lot 824938:3.1±07 μg/mL,
   StemFeed® lot 230438:2.8±0.9 μg/mL.
VEGF concentration measured in the supernatant after 9 days of expansion of patient CD34+ cells ranged from 185.6±18.1 μg/mL (patient 061) to 1032.4±190.4 μg/mL (patient 045).
VEGF concentration measured in the supernatant after 9 days of expansion of healthy donor CD34± cells ranged from 315.3±31.4 μg/mL (FHD_1) to 718.3±54.6 μg/mL (FHD_2).

The average VEGF concentrations obtained from CD34+ cell expansions of 4 healthy donors, 16 patients, and 3 culture media alone (StemFeed® without cytokines) as negative controls are shown in Table 32 below.

TABLE 32
VEGF concentrations obtained from CD34+ cell expansions
of heatlhy donors and patients, and negative controls

| | | | Negative control |
|---|---|---|---|
| Samples | FHD | Patient | StemFeed ® |
| Minimum value | 315.3 | 185.6 | 2.7 |
| Maximum value | 718.3 | 1032.4 | 3.1 |
| Mean (pg/mL) | 516.2 | 596.2 | 2.8 |
| SD | 208.1 | 242.3 | 0.2 |

The mean value of VEGF concentration obtained from 4 healthy donors (FHD) over the 3 trials is 516.2±208.1 μg/mL with a minimum value of 315.3 μg/mL (FHD_1) and a maximum value of 718.3 μg/mL (FHD_2).
The mean value of VEGF concentration obtained from 16 patients is 596.2±242.3 μg/mL with a minimum value of 185.6 μg/mL (patient 061) and maximum of 1032.4 μg/mL (patient 045).
The mean value of VEGF concentration obtained from 3 StemFeed® is 2.8±0.2 μg/mL with a minimum value of 2.7 μg/mL and a maximum of 3.1 μg/mL.

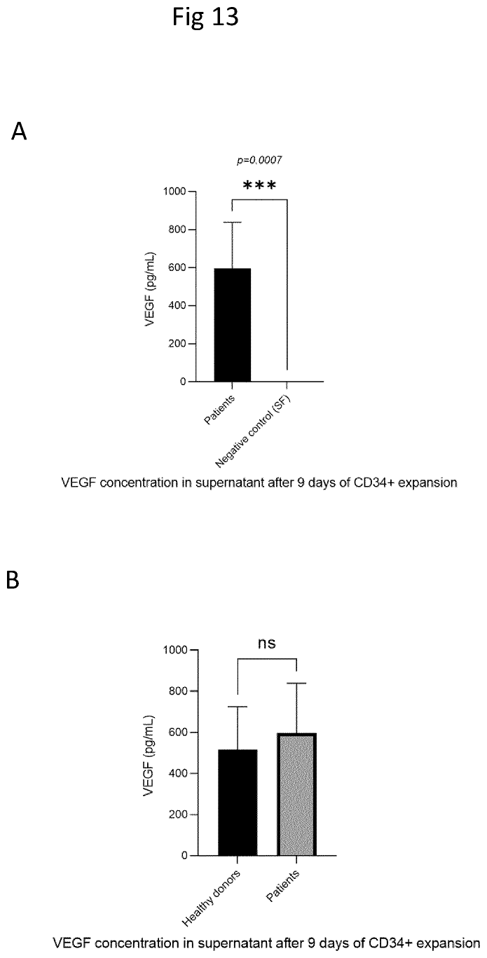

Finally, there is a significant difference between the VEGF concentration in patient supernatant and the VEGF concentration in StemFeed® culture medium alone (t-test, p=0.0007; FIG. 13A), but no significant difference was shown when the VEGF concentration in patient supernatant was compared to the VEGF concentration in healthy donor supernatant (t-test, p=0.5534; FIG. 13B).

Correlation Between VEGF Concentration and Number of CD34+ Cells Obtained after Expansion

Table 33 below shows data from ProtheraCytes® manufacturing from patients in the EXCELLENT study.

TABLE 33
Number of CD34+ cells after 9 days of expansion

| | Number of CD34+ cells |
|---|---|
| ID Patient | after culture (×10⁶) |
| 045 | 182.7 |
| 049 | 44.3 |
| 052 | 36.7 |
| 055 | 64.6 |
| 056 | 41.9 |
| 059 | 256.4 |
| 061 | 29.8 |
| 062 | 165.1 |
| 065 | 211.6 |
| 066 | 73.1 |
| 068 | 62.6 |
| 072 | 75.0 |
| 076 | 79.0 |
| 078 | 226.9 |
| 079 | 58.7 |
| 081 | 101.9 |

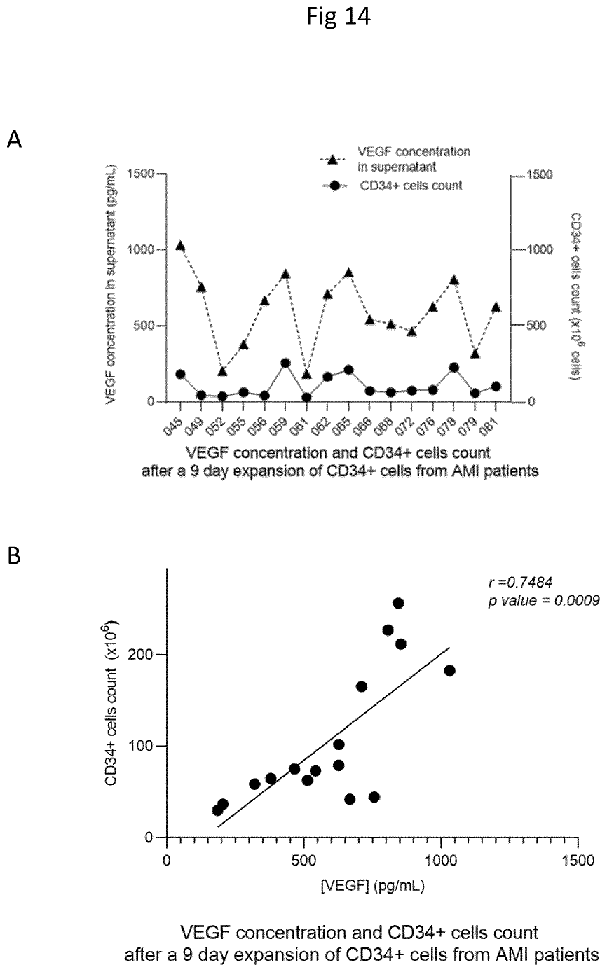

This data is also shown in FIG. 14.

FIG. 14A demonstrates that the curve obtained on the concentration of VEGF in the supernatant mimics the curve from the number of CD34+ cells obtained after 9 days of cell expansion. FIG. 14B shows that there is a positive significant correlation between VEGF concentration and number of CD34+ cells after expansion (Pearson correlation coefficient=0.7484; p value=0.0009).

Finally, statistical analyses show no significant difference (p=0.8686, ANOVA test) when the 3 assays are compared, see Table 34. Shapiro-Wilk/test result: data in bold shown in Table 34 below present p values<0.05 and these data do not follow a normal distribution.

After the Shapiro-Wilk test, data were compared using:
A t-test when data follow a normal distribution.
A Mann-Whitney test when data do not follow a normal distribution.

TABLE 34
Shapiro-Wilk test results

| | Assay 1 | Assay 2 | Assay 3 |
|---|---|---|---|
| Number of values | 16.0 | 16.0 | 16.0 |
| Descriptive statistics | | | |
| Minimum | 190.1 | 165.7 | 201.1 |
| Maximum | 891.1 | 1156.6 | 1127.0 |
| Range | 701.0 | 991.3 | 925.9 |
| Mean | 588.8 | 577.6 | 622.3 |
| Std. Deviation | 237.5 | 257.3 | 248.9 |
| Shapiro-Wilk test | | | |
| P value | 0.1724 | 0.7545 | 0.9155 |
| Passed normality test (alpha = 0.05)? | Yes | Yes | Yes |
| ANOVA test | | | |

| | |
|---|---|
| p value | 0.8686 |

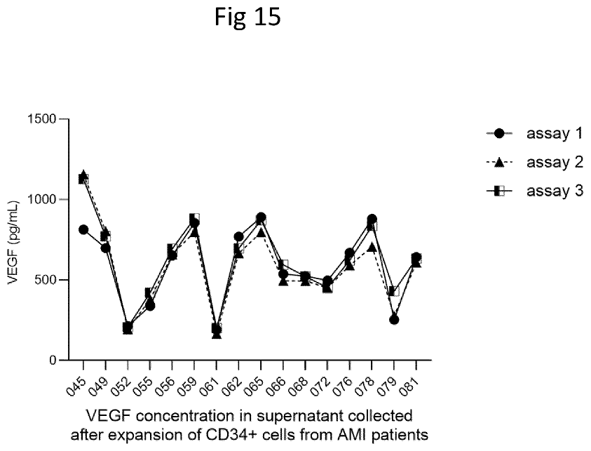

Data curves obtained for the 3 assays are shown in FIG. 15.

### Conclusion

3 trials were performed under the same conditions with the same operator and facilitator to quantify the concentration of VEGF secreted by CD34+ cells in the cell culture supernatants after 9 days of cell expansion. For each sample analysed, an average of these concentrations was calculated.

This quantification of the concentration of VEGF:
secreted by CD34+ cells of:
   16 patients with myocardial infarction (EXCELLENT study),
   4 healthy donors,
in Stemfeed® culture medium before expansion (as a negative control), showed that:
   The concentration of secreted VEGF in the culture supernatant of CD34+ cells from patients ranged from 185.6 μg/mL to 1032.4 μg/mL with a mean value of 596.2±242.3 μg/mL,
   The concentration of secreted VEGF in the culture supernatant of healthy donor cells ranged from 315.3 μg/mL to 718.3 μg/mL with a mean value of 526.2±208.1 μg/mL,
   The concentration of VEGF observed in the StemFeed® culture medium before expansion, (negative control) varies from 2.7 μg/mL to 3.0 μg/mL with a mean value of 2.8±0.2 μg/mL.

These results show that the VEGF concentration observed in the supernatant after expansion of CD34+ cells from patients (mean value of 596.2±242.3 μg/mL) is high:
no significant difference was observed when this concentration was compared with the VEGF concentration in the supernatant of CD34+ cells from healthy donors (mean value of 526.2±208.1 pg/mL),
a significant difference (t-test, p=0.0007) was observed when this concentration was compared to that quantified in StemFeed® culture medium alone (2.8±0.2 μg/mL).

Furthermore, the concentration of VEGF in the culture supernatant of the patients' CD34+ cells was significantly correlated with the number of CD34+ cells obtained after expansion (Pearson correlation coefficient r=0.7484; p-value=0.0009), thus supporting the secretion of VEGF by these CD34+ cells.

Finally, statistical analyses show no significant difference (ANOVA test p=0.8686) when the 3 assays are compared.

### Correlation Between Amount of VEGF Secreted Per Cell and Clinical Endpoints

### Method

The on-going EXCELLENT trial (EUDRACT 2014-001476-63) investigates the use of autologous peripheral blood (PB)-CD34+ cells, isolated from acute myocardial infarction (AMI) patients, expanded by the automated StemXpand® device and StemPack® production kits developed by CellProthera, and injected trans-endocardially.

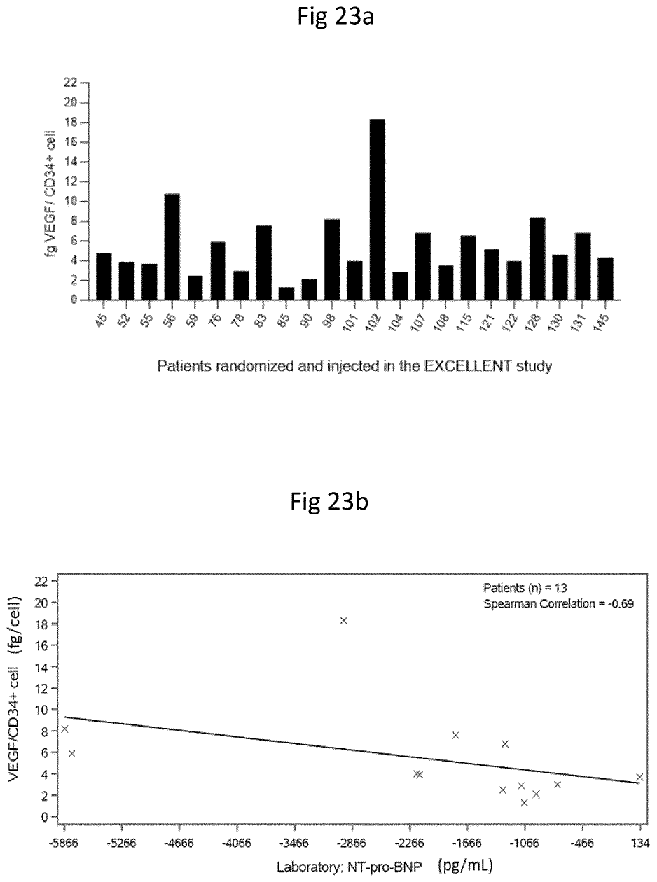

The amount of VEGF secreted by CD34+ cells isolated from 13 patients in the trial was measured (see FIG. 23a). For the same 13 patients the change in NT-proBNP at 6 months following administration of CD34+ cells compared to baseline was measured.

Spearman rank correlations were performed between the amount of VEGF secreted per cell (fg/cell) and the interim analysis of different clinical endpoints from the EXCELLENT clinical study.

### Results

A significant negative correlation was observed between the VEGF secretion/cell (fg/cell) and N-terminal prohormone of brain natriuretic peptide (NT-proBNP) (pg/mL), which is a predictor of death, cardiovascular events, and heart failure. Spearman Correlation (95% CI)=−0.69 (−0.90; −0.22); p value=0.0057 (see FIG. 23b). These results show that higher VEGF secretion by CD34+ cells (ProtheraCytes) is correlated with lower NT-proBNP and improvement in AMI patients. This indicates that VEGF is useful as an indicator of CD34+ cell potency and can be used as marker to select CD34+ cells for therapy. Furthermore, all patients who received autologous expanded CD34+ cells that secreted at least 1.3 fg/cell VEGF exhibited an improvement in NT-proBNP levels. For example, even the patient who had the lowest secretion of VEGF (1.3 fg/cell), had a decrease of 1066 μg/ml in NT-proBNP at 6 months compared to baseline. This indicates that CD34+ cells secreting VEGF of the order of around 1 fg/cell are therapeutically beneficial.

### Example 3: MicroRNA Studies

The objective of this technical example was to analyze the expression of the following miRNAs in CD34+ cells after 9 days of culture (ProtheraCytes®) versus exosomes from ProtheraCytes® (exosomes: nanovesicles produced by ProtheraCytes®) from 7 patients from the EXCELLENT clinical study (062, 065, 066, 068, 072, 079, 081):
Proangioangenic miRNAs such as miR126, miR130a, miR21, miR26a, miR378a
Antiapoptotic miRNAs such as miR146a, miR21
miRNAs increasing proliferation of cardiomyocytes: miR199a, miR590
Antifibrotic miRNA such as miR133a

### Materials and Methods

### Exosome Production Protocol

Cells were cultured in conditioned medium of cytokine cocktail and StemSpan-AOF (StemCell Technologies, BC Canada, ref: 100-0130).

Culture of Cells from Frozen Cells

Under a laminar flow hood, a 50 ml tube was prepared with 20 ml of StemSpan-AOF medium. Cells were removed from liquid nitrogen and placed on ice, before thawing in a 37° C. water bath. As soon as the cells were thawed, the cell vial was disinfected with 70% alcohol and cells were transferred into the 50 mL tube containing 20 ml of StemSpan-AOF medium. The tubes were centrifuged at 300 g for 10 minutes.

Under a laminar flow hood, the supernatant was removed and the cell pellet resuspended in 1 ml of conditioned medium. 2.5×10⁶ cells were cultured in T25 flasks in 10 ml of conditioned medium and incubated at 37° C., 5% CO2 for 40-48 h.

Culture of Cells from Fresh Cells

Tubes containing fresh cells were centrifuged at 400 g for 10 minutes. Under a laminar flow hood, the supernatant was removed and the cell pellet resuspended in 1 mL of conditioned medium. 2.5×10⁶ cells were cultured in T25 flasks in 10 mL of conditioned medium and incubated at 37° C., 5% CO₂ for 40-48 h.

### Protocol for Purification of Exosomes by Precipitation

Exosomes were purified by precipitation from ExoQuick-TC™ (System Biosciences, CA, USA, ref: EXOTC50A-1).
1. Centrifuge at 3000 g for 15 minutes, 10 mL cell suspension (to remove cells and debris)
2. Transfer supernatant in a 15 mL new tube and add 2 mL of ExoQuick-Tc solution
3. Keep the pellet for microRNA extraction: resuspend the pellet in 260 μL of RTL buffer from the miRNeasy Tissue/Cells Advanced Mini Kit (Qiagen, ref: 217604).
4. Mix the tube by inversion
5. Incubate overnight (at least 12 hours) at +2-8° C. (do not stir/mix the tube during incubation, the tube should remain upright
6. The day after, centrifuge the tube at 1500 g for 30 minutes at room temperature (15-25° C.)
7. After centrifugation, the exosomes appear as a white/beige pellet
8. Remove the supernatant (aspirate all trace of solution)
9. MicroRNA extraction from exosomes: resuspend the pellet in 200 μL of buffer of resuspension from Qiagen protocol miRNeasy Serum/Plasma or miRNeasy (Qiagen, ref: 217204)
10. For flow cytometry analysis, resuspend the cell pellet in the appropriate volume of sterile PBS (300-500 L)
   Protocol for labelling exosomes in flow cytometry

### I. Preparation of the Control: “Standard_Exosomes” Sample

Reconstitution of exosomes: recommended to reconstitute the standard with sterile water to a final concentration of 1 μg/μL.
For 100 μg of standard, add 100 μL of sterile water
Vortex and centrifuge briefly.
Make aliquots of 5-7 μL
Store at −80° C.

### II. CD63+/CD81+/CD34+ Exosome Labelling—Day 1

1. Prepare the starting suspension of the Standard Exosomes control:
   a) 5 μL of standard exosomes+95 μL of water for injection for a final concentration of 0.05 μg/μL
2. Resuspend the CD63 capture beads by vortexing for about 20 seconds
3. Add 50 μL of CD63 capture beads to:
   a) Tube 1 «Background noise_CD63 capture beads»
   b) Tube 2«control_Exo Standard» CD81FITC (indirect labelling)
   c) Tube 3-1: «Exo ProtheraCytes» CD81FITC (indirect labelling)
d) Tube 3-2: «Exo ProtheraCytes» CD34 PE (direct labelling)
e) Tube 3-3: «Exo ProtheraCytes» CD81FITC (indirect labelling) CD34 PE (direct labelling)
f) Tube 4-1: «Exo Fraction neg» CD81FITC (indirect labelling)
g) Tube 4-2: «Exo Fraction neg» CD34 PE (direct labelling)
h) Tube 4-3: «Exo Fraction neg» CD81FITC (indirect labelling) CD34 PE (direct labelling)
4. Resuspend the IgG1 capture beads: by vortexing for about 20 seconds and add 50 μL of “IgG1 capture beads” to 2 FACS tubes:
   a) Tube 5=>Exo ProtheraCytes®_IgG1-PE (direct labelling)
   b) Tube 6=>Exo ProtheraCytes® _IgG1-FITC (indirect labelling)
5. Prepare the tubes Compensations: Add 50 μL of CD63 capture beads in:
   a) Tube 7=> (CD63 beads+Exo ProtheraCytes®)
   b) Tube 8=> (CD63 beads+Exo ProtheraCytes®+CD81 FITC)
   c) Tube 9=> (CD63 beads+Exo ProtheraCytes®+34 PE)
6. Add 100 μL of exosome suspension to the:
   a) Tube 2=>Exo Standard
   b) Tubes 3=>Exo ProtheraCytes®
   c) Tubes 4=>Exo Fraction neg
   d) Tubes 5 et 6=>Exo ProtheraCytes®
   e) Tube 7=>Exo ProtheraCytes®
   f) Tube 8=>Add 100 μL Exo ProtheraCytes®
g) Tube 9=>Add 100 L Exo ProtheraCytes®
7. Mix the suspension by pipetting gently several times and vortexing for a few seconds
8. Incubate in the dark at room temperature (RT) overnight

### III. CD63+/CD81+/CD34+ Exosome Labelling-Day 2

9. Add antibodies for identification as follows:
   a) Tube 2: Add 5 L CD81-biotin (exo standard) (indirect labelling)
   b) Tube 3-1: Add 5 μL CD81-biotin (exo ProtheraCytes®) (indirect labelling)
   c) Tube 3-2: Add 5 μL CD34-PE (exo ProtheraCytes®) (direct labelling)
   d) Tube 3-3: Add 5 μL CD81-biotin and 5 μL CD34-PE (exo ProtheraCytes®) (indirect and direct labelling)
   e) Tube 4-1: Add 5 μL CD81-biotin (exo Fraction neg) (indirect labelling)
   f) Tube 4-2: Add 5 μL CD34-PE (exo Fraction neg) (direct labelling)
   g) Tube 4-3: Add 5 μL CD81-biotin and 5 μL CD34-PE (exo Fraction neg) (indirect and direct labelling)
   h) Tube 5: Add 5 μL IgG1-PE (direct labelling)
   i) Tube 6: Wait for secondary labelling (indirect IgG FITC labelling)
   j) Tube 8: Add 5 μL CD81-biotin (exo ProtheraCytes®) (indirect labelling)
   k) Tube 9: Add 5 μL CD34-PE (exo ProtheraCytes®) (direct labelling)
10. Mix by gently tapping the tube
11. Incubate 1 hour at +2-8° C. in the dark
12. Wash with 1 mL of 1X Assay Buffer
13. Centrifuge tubes at 2500 g, 5 minutes at +4° C.
14. Gently remove the supernatant leaving about 100 μL at the bottom of the tube (with a p1000 pipette, gently remove 1 mL)
15. Resuspend exosomes and beads by adding:
   i. For direct labelling tubes: 150 μL “Assay Buffer 1× (ImmunoStep, Spain)
   ii. For 2^{dary} labelling:
      1. Add 5 μL streptavidin-FITC in tubes:
         a. 2
         b. 3-1
         c. 3-3
         d. 4-1
         e. 4-3
         f. 6
         g. 8
16. Incubate 30 minutes at +2-8° C. in the dark
17. Wash by adding 1 mL of Assay Buffer 1×
18. Centrifuge the tubes at 2500 g, 5 minutes at +4° C.
19. Gently remove the supernatant leaving about 100 μL at the bottom of the tube (with a p1000 pipette, gently remove 1 mL)
20. Resuspend exosomes and beads by adding 150 μL of Assay Buffer 1×
21. Proceed with the acquisition on the flow cytometer in medium (can wait a maximum of 2 hours at +2-8° C. before acquisition)
   Protocol for microRNA Analysis in RT-qPCR

### Exosomes Produced by ProtheraCytes®:

### a) MicroRNA Extraction (RNeasy Qiagen®)

1. Transfer 200 μL serum or plasma into a 2 mL tube
2. Add 60 μL Buffer RPL. Close the tube caps and vortex for >5 s. Incubate at room temperature for 3 min.
3. Add 20 μL Buffer RPP. Close the tube caps and mix vigorously by vortexing for >20 s. Incubate at room temperature for 3 min.
4. Centrifuge at 12000×g for 3 min at room temperature to pellet the precipitate. Note: Supernatant should be clear and colourless.
5. Transfer the supernatant (˜230 μL) to a new reaction tube. Add 1 volume isopropanol. Mix well by vortexing. Transfer the entire sample to an RNeasy UCP MinElute column. Close the lid, and centrifuge for 15 s at >8000×g. Discard the flow-through.
6. Pipet 700 μL Buffer RWT to the RNeasy UCP MinElute spin column. Close the lid, and centrifuge for 15 s at ≥8000×g. Discard the flow-through.
7. Pipet 500 μL Buffer RPE onto the RNeasy UCP MinElute spin column. Close the lid, and centrifuge for 15 s at >8000×g. Discard the flow-through.
8. Add 500 μL of 80% ethanol to the RNeasy UCP MinElute spin column. Close the lid, and centrifuge for 2 min at >8000×g. Discard the flow-through and the collection tube.
9. Place the RNeasy UCP MinELute spin column in a new 2 mL collection tube. Open the lid of the spin column and centrifuge at full speed for 5 min to dry the membrane. Discard the flow-through and the collection tube.
10. Place the RNeasy UCP MinElute spin column in a new 1.5 mL collection tube. Add 20 μL RNase-free water directly to the center of the spin column membrane and incubate 1 min. Close the lid, and centrifuge for 1 min at full speed to elute the RNA.
11. Store RNA at −80° C.
   b) First Strand cDNA Synthesis
Rq: the RNA spike-in tube for RT is an internal extraction and amplification control
Before starting:
   Thaw on ice
      RNA samples
      5× miRCURY RT SYBR Green Reaction buffer
   Put in suspension
      RNA spike-in: UnuSp6 spike in 80 μL RNase free water
      Vortex and centrifuge briefly, incubate 20-30 min on ice
      Vortex and centrifuge briefly, aliquot and store at −20° C.
   Remove 10× miR CURY Rt Enzyme tube from freezer
      Mix gently, place on ice, centrifuge briefly, keep on ice

### 1) Calculate the Volume of RNA Sample

$$RNA sample [ μl ] = elution Vol [ μl ] / starting sample Vol * 16 [ μl ] ( 1.6 = 20 / 200 * 16 )$$

### 2) Prepare the Reverse Transcription According to Table 35 Below.

TABLE 35
Reverse Transcription reaction reagents

| Reagents | miRNA Exosome (μL) |
|---|---|
| 5X miRCURY SYBR Green RT | 2 |
| Reaction Buffer | |
| RNAse free water | 4.9 |
| 10x miRCURY RT Enzyme Mix | 1 |
| UniSp6 RNA spike-in | 0.5 |
| RNA sample | 1.6 |
| Total reaction volume | 10 |

### Rt Program:

60 min @ 42° C.
5 min @ 95° C.
Infinite @ 4° C.

### Freeze @−20° C.

### c) Quantitative Real-Time PCR

Resuspend the miRCURY LNA PCR Assays tubes below for the 1st time:
   UniSp6 Spike-in control PCR Assays
   hsa-mi-103a-3p,
   miR-130a-3p,
   miR126-3p
   Centrifuge before opening, add 220 μL of RNAse free water, 20 min at room temperature, Vortex and centrifuge
Thaw the following tubes
   2× miRCURY SYBR Green Master Mix,
   cDNA,
   LNA PCR Assays tubes (Spike-in primer Sp6, mi 103a-3p, miR130a-3p, miR126-3p, H₂O RNAse free
1. Dilute cDNA 1:30 (add 290 μL of RNAse free water to 10 μL reverse transcription reaction)
2. Prepare a reaction mixture according to Table 36 below.

TABLE 36
Reaction mixture for quantitative real-time PCR

| Reagents | Vol/1 tube (uL) |
|---|---|
| 2X miRCURY SYBR Green | 5 |
| Master Mix | |
| Mix Primer | 1 |
| RNAse free water | 1 |
| Total | 7 |

3. Add:
   3 μL of RT product (diluted 1:30) in each well of 1 PCR plate,
   7 μL of Mix/well
4. Briefly centrifuge the tubes or plate at room temperature
5. Program the CFX96 according to the Table 37 below.

TABLE 37
PCR cycle conditions for PCTmiCURY LNA miRNA assays

| | | | |
|---|---|---|---|
| 2 min | @ 95° C. | | PCR initial heat |
| | | | activation |
| 10 s | @ 95° C. | 40 cycles | Denaturation |
| 60 s | @ 56° C. | | Elongation |
| | 60-95° C. | | Melting curve analysis |

6. Place the PCR plate in the CFX96 and start the program
7. Perform the initial analysis using the CFX96 software to obtain the raw Cq values (Cq or CT depending on the PCR device)

### ProtheraCytes®

a) microRNA Extraction
miRNA Cells

### RLT Buffer (Qiagen®)

260 UL pellet <5×10⁶ cells
450 μL pellet ≥5×10⁶ cells
Homogenize by vortexing and pipetting several times, storage at −80° C.

If necessary thaw the sample,
Homogenize by vortexing and pipetting several times

### QIAshredder Column (Qiagen®)

Deposit the lysate
Centrifuge for 2 minutes at 12000 g at room temperature

### Add AL Buffer

80 μL pellet <5×10⁶ cells
140 μL pellet ≥5×10⁶ cells
Mix by vortexing strongly and incubate for 3 min at 20° C.
Place the eluate on the gDNA Eliminator column with its 2 mL collection tube
Centrifuge for 30 sec at 8000 g
Add 1 volume of Isopropanol (340 μL or 590 μL)
Mix by pipetting, do not spin
Place a maximum of 700 μL of filtrate+ethanol on the RNeasy column
Centrifuge 15 sec at 8000 g at 20° C.
Discard eluate
Repeat the operation if necessary
Apply 700 μL of RWT buffer
Centrifuge 15 sec at 8000 g at 20° C.
Place the column on a new tube
Add 500 μL of RPE buffer
Centrifuge 15 sec at 8000 g at 20° C.
Place the column on a new tube
Add 500 μL of 80% ethanol
Centrifuge for 2 min at 8000 g at 20° C.
Place the column on a new tube
Centrifuge for 1 min at 12000 g to elute the RNA
Place the column on a new sterile 1.5 mL microtube
Add 35 μL of RNAse free water
Incubate 1 min at room temperature
Centrifuge for 1 min at 12000 g to elute the RNA
Run the eluate back through the RNeasy column
Centrifuge for 1 min at 8000 g at 80° C.
Store RNA at −80° C.
   b) First Strand cDNA Synthesis
Optimised protocol for use with 20 ng RNA in reverse transcription
For highly expressed miRNAs: use up to 10 μg of total RNA
For low expressed miRNAs: use up to 200 ng total RNA

Before starting:
Thawing on ice
   RNA samples
   5×miRCURY RT SYBR Green reaction Buffer
Thaw RNAse free water on ice at room temperature
   Mix each solution by scraping the tubes
   Vortex and centrifuge briefly; incubate 20-30 min on ice
Put in suspension
RNA spike-in: UniSp6 RNA spike in 80 μL of RNAse free water
   Vortex and centrifuge briefly, incubate 20-30 min on ice
   Vortex and centrifuge briefly, aliquot and store at −20° C.
Remove the 10× miR CURY RT Enzyme tube from the freezer
   Mix gently, place on ice, centrifuge briefly, keep on ice
1) Dilute RNA sample to 5 ng/μL RNAase free water
2) Prepare the RT according to Table 38 below.

TABLE 38
Reverse Transcription reaction reagents

| Reagents | miRNA PCR (μL) |
|---|---|
| 5X miRCURY SYBR Green RT | 2 |
| Reaction Buffer | |
| RNAse free water | 4.5 |
| 10x miRCURY RT Enzyme Mix | 1 |
| UniSp6 RNA spike-in | 0.5 |
| RNA sample | 2 |
| Total reaction volume | 10 |

### Rt Program:

60 min @ 42° C.
5 min @ 95° C.
Infinite @ 4° C.

### c) Quantitative Real-Time PCR

Resuspend the 1st time, miRCURY LNA miRNA PCR Assays tubes below:
   UniSp6-Spikelin control PCR Assays
   hsa-miR-103a-3p
   miR130a-3p
   miR126-3p
Centrifuge before opening, add 220 μL of RNAse free water, 20 min at room temperature
Vortex and centrifuge briefly
Thaw the following tubes
   2× miRCURY SYBR Green Master Mix
   CDNA
   LNA PCR Assays
   H₂O RNase free
1) Dilute cDNA 1:60 (add 590 μL RNase free water to 10 μL RT reaction)
2) Prepare a reaction mix according to Table 39 below.

TABLE 39
Reverse Transcription reaction reagents

| Reagents | Vol/1 tube (μL) |
|---|---|
| 2X miRCURY SYBR Green | 5 |
| Master Mix | |
| Mix Primer | 1 |
| RNAse free water | 1 |
| Total | 7 |

3) Add:
   3 μL of RT product (diluted 1:60) in each well of 1 PCR plate
   7 μL of Mix/well
4) Briefly centrifuge the tubes or plate at room temperature
5) Program the CFX96 according to Table 40 below

TABLE 40
PCR cycle conditions for PCTmiCURY LNA miRNA assays

| | | | |
|---|---|---|---|
| 2 min | @ 95° C. | | PCR initial heat |
| | | | activation |
| 10 s | @ 95° C. | 40 cycles | Denaturation |
| 60 s | @ 56° C. | | Elongation |
| | 60-95° C. | | Melting curve analysis |

6. Place the PCR plate in the CFX96 and start the program
7. Perform the initial analysis using the CFX96 software to obtain the raw Cq values (Cq or CT depending on the PCR device)

### Summary of Tests Carried Out

TABLE 41A
Summary table of the tests carried out

| | MicroRNA | MicroRNA | MicroRNA | MicroRNA study 7 | MicroRNA |
|---|---|---|---|---|---|
| | study 4 (FHD) | study 5 (FHD) | study 6 (FHD) | (patient 081) | study 8 (FHD) |
| Date of the | From February | From February 4 | From March 18 | From April 1 | From April 8 |
| assay | 18 to 22, 2022 | to 8, 2022 | to 22, 2022 | to 5, 2022 | to 12, 2022 |
| Cells from the | Essai_Cytokines | Essai_Cytokines | Essai_Cytokines | Positive and | Essai_Stab_SF |
| assay | comparaison - 1 - | comparaison - 2 - | comparaison - 3 - | negative fractions | 279511 T0M |
| | K0321 - I2120 | K0321 - I2120 | K0321 - I2120 | of Patient 081/04 | |
| | | | | from the | |
| | | | | EXCELLENT study | |

TABLE 41B
Summary table of the tests carried out

| | MicroRNA study 9 (patients 062; |
|---|---|
| | 065; 066; 068; 072; 079) |
| Date of the assay | May 9 to 12, 2022 (exosome |
| | production part) |
| Cells from assay | Positive fractions frozen - |
| | patients study EXCELLENT |

### Results

Flow Cytometry Analysis—Exosomes from Experimental Studies 4, 5, 6, 7 and 8

TABLE 42
Flow cytometry analysis-exosomes from experimental studies 4, 5, 6, 7 and 8

| | Flow cytometry analysis on exosomes from positive and negative fractions | | | | | | | | | |
|---|---|---|---|---|---|---|---|---|---|---|
| | MicroRNA | | MicroRNA | | MicroRNA | | MicroRNA | | MicroRNA | |
| | study 4 | | study 5 | | study 6 | | study 7 | | study 8 | |
| | FHD | | FHD | | FHD | | Patient 081 | | FHD | |
| | Positive | Negative | Positive | Negative | Positive | Negative | Positive | Negative | Positive | Negative |
| | fraction | fraction | fraction | fraction | fraction | fraction | fraction | fraction | fraction | fraction |
| % CD63CD81 | 81.2 | 75.0 | 50.3 | 53.9 | 25.3 | 39.2 | 83.6 | 42.7 | 37.0 | 10.5 |
| % CD34 | 17.9 | 3.1 | 8.4 | 4.7 | 22.6 | 5.2 | 29.5 | 6.1 | 19.6 | 2.2 |

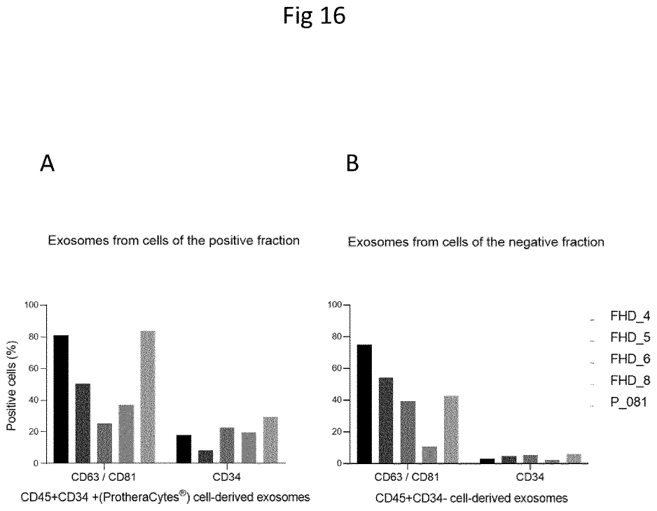

Exosomes from ProtheraCytes® (positive fraction) express exosome-specific membrane markers (CD63, CD81), as well as the CD34 marker of the cell from which they originate (FIG. 16A). As expected, the exosomes of the negative fraction express low levels of the CD34 marker (FIG. 16B).

### Proangiogenic MicroRNA Expression in CD34+ Cell-Derived Exosomes Versus Cells

### Housekeeping Gene Expression:

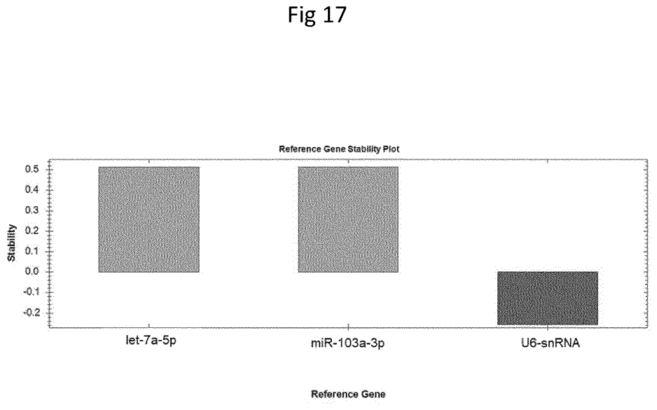

3 housekeeping genes (miR-103a, let7a-5p and U6) were analyzed to determine which one is the best for the analysis of miRNA of interest. The results can be seen in FIG. 17, demonstrating that U6 is unstable, therefore this housekeeping gene was not retained for this analysis.

### Proangiogenic MicroRNA Expression

I. Results from Patient 081 from EXCELLENT Study-Experimental Study 7:

3 proangiogenic miRNAs were analyzed:
miRNA126
miRNA130a
miRNA378a

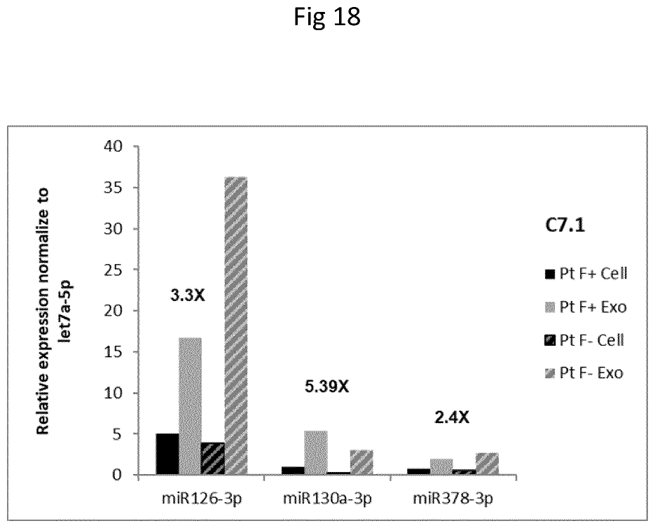

The results of proangiogenic miRNA's in ProtheraCytes® exosomes from AMI patient 081 are shown in FIG. 18, wherein the figure key is:

$$Pt F \text{+} Cell = Patient\_Positive Fraction\_Cells$$

$$Pt F + Cell = Patient\_Positive Fraction\_Exosomes$$

$$Pt F - Cell = Patient\_Negative Fraction\_Cells$$

$$Pt F - Cell = Patient\_Negative Fraction\_Exosomes$$

The proangiogenic miRNAs 126, 130a, 378a are more expressed in ProtheraCytes® exosomes compared to cells.

Analysis with the let7a-5p housekeeping gene showed a better result in terms of microRNA expression and was kept for the analysis. In the positive fraction, for the same microRNA, when its expression in exosomes (Pt F+Exo) is compared to the expression of this microRNA in cells (Pt F+ Cell), the results are as follows:
miRNA 126 is expressed 3.3-fold more in exosomes compared to cells.
miRNA 130a is expressed 5.4-fold more in exosomes compared to cells.
miRNA 378a is expressed 2.4-fold more in exosomes compared to cells

These results are in line with previously published results by Sahoo et al. 2011: “Exosomes From Human CD34+ Stem Cells Mediate Their Proangiogenic Paracrine Activity” (Circ Res., 2011 Sep. 16; 109(7):724-8).

II. Results from Healthy Donors (FHD) from Experimental Study 4, 5, 6 and 8

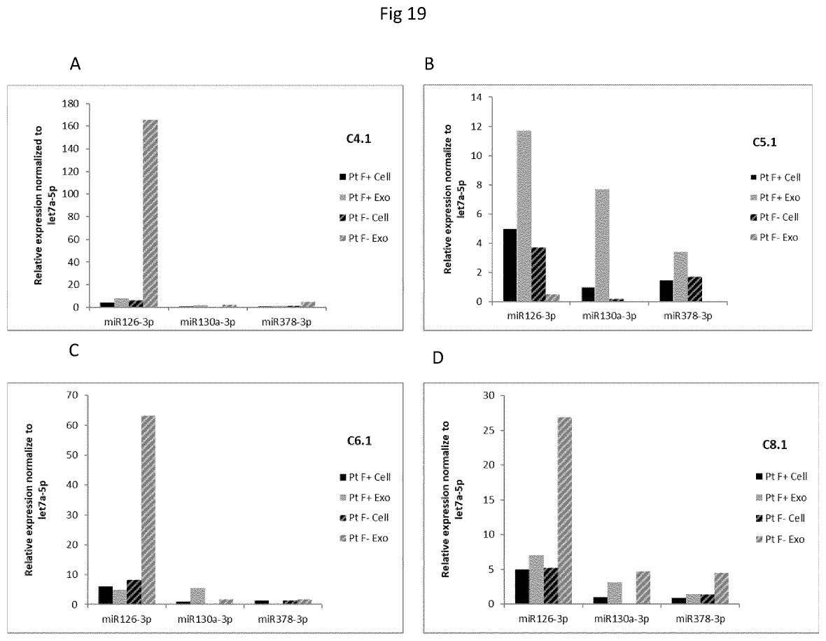

The results of proangiogenic miRNA's in ProtheraCytes® exosomes from healthy donors (FHD)—experimental studies 4, 5, 6 and 8 are shown in FIG. 19, wherein the figure key is:

$$Pt F \text{+} Cell = Patient\_Positive Fraction\_Cells$$

$$Pt F + Cell = Patient\_Positive Fraction\_Exosomes$$

$$Pt F - Cell = Patient\_Negative Fraction\_Cells$$

$$Pt F - Cell = Patient\_Negative Fraction\_Exosomes$$

C4.1-MicroRNA study 4_FHD (FIG. 19A)
   Essai_Cytokines comparison-1-K0321-12120
C5.1-MicroRNA study 5_FHD (FIG. 19B)
   Essai_Cytokines comparison-2-K0321-12120
C6.1-MicroRNA study 6_FHD (FIG. 19C)
   Essai_Cytokines comparaison-3-K0321-12120
C8.1-MicroRNA study 8_FHD (FIG. 19D)
   Essai_Stab_SF 279511 T0M

Finally, analysis with the let7a-5p housekeeping gene of the average microRNA expressions (FHD+patient) showed in the positive fraction for the same microRNA, that when its expression in exosomes is compared to the expression of this microRNA in cells, the result is the following:
No difference in expression for miRNA 126
miRNA 130a is expressed 3.8-fold more in exosomes compared to cells
miRNA 378a is expressed 1.8-fold more in exosomes compared to cells

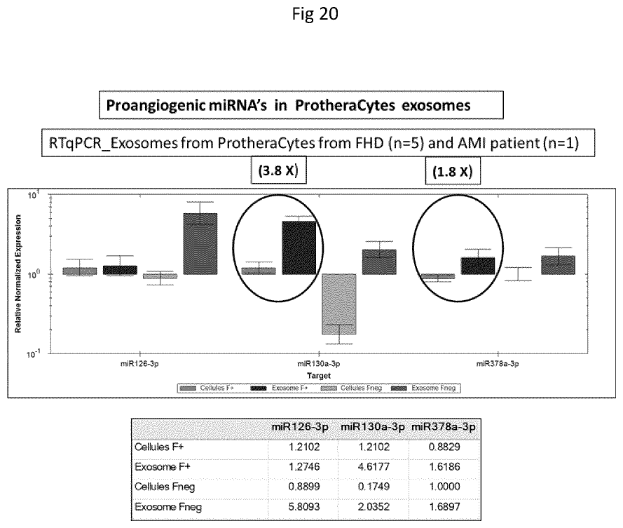

This data, of proangiogenic miRNA's in ProtheraCytes® exosomes is shown in FIG. 20 and Table 43 below, with RTqPCT_Exosome from ProtheraCytes® from FHD (n=5) and AMI patient (n=1).

Figure key for FIG. 20 is as follows:

$$Pt F \text{+} Cell = Patient\_Positive Fraction\_Cells$$

$$Pt F + Cell = Patient\_Positive Fraction\_Exosomes$$

$$Pt F - Cell = Patient\_Negative Fraction\_Cells$$

$$Pt F - Cell = Patient\_Negative Fraction\_Exosomes$$

TABLE 43
Proangiogenic miRNA's in ProtheraCytes ® exosomes
- RTqPCT_Exosome from ProtheraCytes ® from
FHD (n = 5) and AMI patient (n = 1).

| | miR126-3p | miR130a-3p | miR378a-3p |
|---|---|---|---|
| Cells F+ | 1.2102 | 1.2102 | 0.8829 |
| Exosome F+ | 1.2746 | 4.6177 | 1.6186 |
| Cells F− | 0.8899 | 0.1749 | 1.0000 |
| Exosome F− | 5.8093 | 2.0352 | 1.6897 |

III. MicroRNA Results from Patients 062, 065, 066, 068, 072, 079 and 081 (EXCELLENT Study)—Experimental Study 9

Previous studies have shown that human adult CD34+ cells secrete exosomes containing high levels of proangiogenic microRNAs, such as the proangiogenic microRNAs 126 and 130a (Sahoo et al, 2011).

To investigate this, expression of the following miRNAs in CD34+ cells after 9 days of culture (ProtheraCytes®) versus exosomes from ProtheraCytes® (exosomes: nanovesicles produced by ProtheraCytes®) from 7 patients (062, 065, 066, 068, 072, 079, 081) was analysed:
Proangioangenic miRNAs: miR126-3p, miR130a-3p, miR21, miR26a, miR378a
Antiapoptotic effect of miRNAs: miR146a, miR21
miRNAs increasing proliferation of cardiomyocytes: miR199a, miR590
Antifibrotic miRNA: miR133a

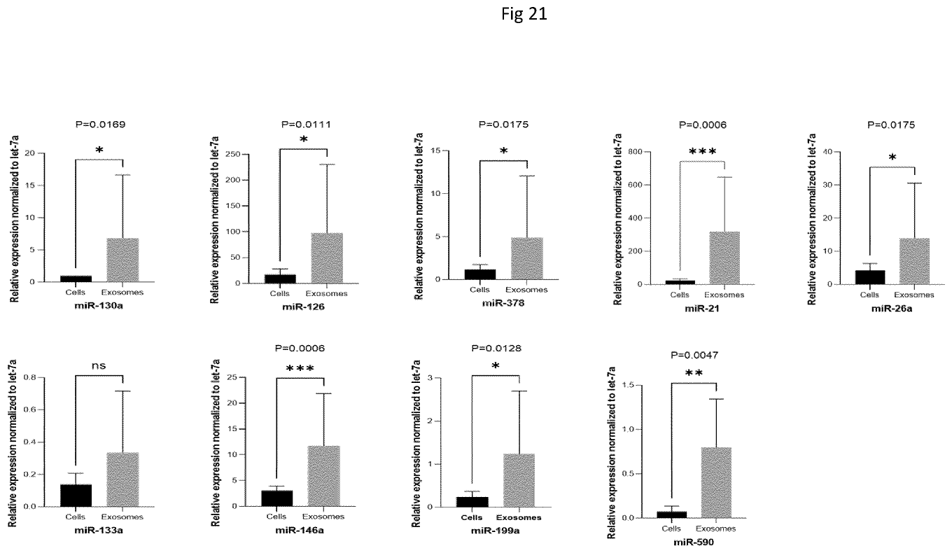

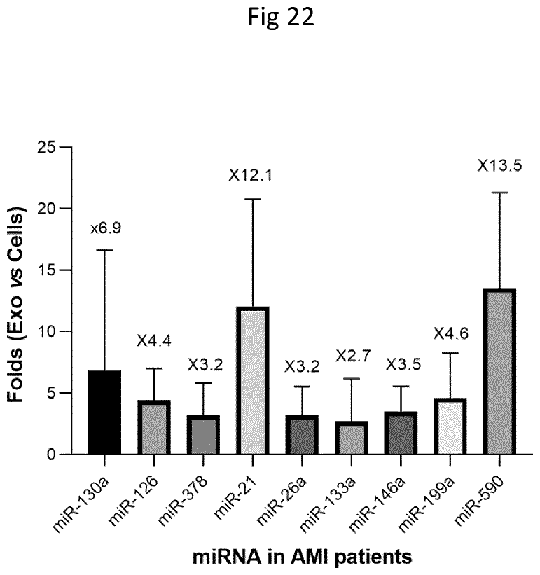

MicroRNA results are shown in the FIGS. 21 and 22.

Comparison of the average miRNA expression obtained in the exosomes produced by these ProtheraCytes® with that obtained in the ProtheraCytes® of the 7 patients analyzed (FIG. 22) shows that:
miR-130a is 6.9 times more expressed in exosomes
miR-126 is 4.4 times more expressed in exosomes
miR-378a is 3.2 times more expressed in exosomes
miR-21 is 12.1 times more expressed in exosomes
miR-26a is 3.2 times more expressed in exosomes
miR-133a is 2.7 times more expressed in exosomes
miR-146a is 3.5 times more expressed in exosomes
miR-199a is 4.6 times more expressed in exosomes
miR-590 is 13.5 times more expressed in exosomes

### Conclusion

Proangiogenic, antiapoptotic, and other miRNAs playing a role in myocardial regeneration are expressed in expanded CD34+ cell-derived exosomes from Acute Myocardial Infarction (AMI) Patients. A growing amount of evidence has demonstrated that miRNAs play an essential role in myocardial regeneration.

miRNAs function in cardiac repair by regulating angiogenesis, proliferation, apoptosis, and metabolism. Previous studies have shown that human adult CD34+ stem cells secreted exosomes contain high levels of proangiogenic microRNAs, such as the proangiogenic microRNAs 126 and 130a (Sahoo et al, 2011)

To investigate this, the expression of the following miRNAs were analyzed in CD34+ cells after 9 days of culture (ProtheraCytes®) versus exosomes from ProtheraCytes® (exosomes: nanovesicles produced by ProtheraCytes®) from 7 patients from the EXCELLENT clinical study (062, 065, 066, 068, 072, 079, 081):
Proangioangenic miRNAs such as miR126, miR130a, miR21, miR26a, miR378a.
Antiapoptotic miRNAs such as miR146a, miR21
miRNAs increasing proliferation of cardiomyocytes: miR199a, miR590
Antifibrotic miRNA such as miR133a

The results have shown that exosomes from ProtheraCytes® have a significantly higher expression of all miRNAs analyzed than ProtheraCytes® cells except for miR133a. The expression of miR-126, miR-130a, miR-21, miR-26 and miR-378a confirm the proangiogenic effect of ProtheraCytes®-derived exosomes.

These results indicate that ProtheraCytes® are able to secrete exosomes containing proangiogenic miRNAs which might lead to the induction of angiogenesis and contribute to the vascular repair process after AMI. These results further suggest that ProtheraCytes® might protect cardiomyocytes from apoptosis immediately after AMI through the secretion of exosomes containing multiple anti-apopototic miRNAs.

### REFERENCES

1. Pasquet S, Sovalat H, Henon P et al. Long-term benefit of intracardiac delivery of autologous granulocyte-colony-stimulating factormobilized blood CD34+ cells containing cardiac progenitors on regional heart structure and function after myocardial infarct. Cytotherapy 2009; 11:1002-1015.
2. Sahoo S, Klychko E, Thorne T, Misener S, Schultz K M, Millay M, Ito A, Liu T, Kamide C, Agrawal H, Perlman H, Qin G, Kishore R, Losordo D W. Exosomes from human CD34 (+) stem cells mediate their proangiogenic paracrine activity. Circ Res. 2011; 109(7):724-8.
3. Saucourt C, Vogt S, Merlin A, Valat C, Criquet A, Harmand L, Birebent B, Rouard H, Himmelspach C, Jeandidier É, Chartois-Leauté A G, Derenne S, Koehl L, Salem J E, Hulot J S, Tancredi C, Aries A, Jude S, Martel E, Richard S, Douay L, Henon P. Design and Validation of an Automated Process for the Expansion of Peripheral Blood-Derived CD34+ Cells for Clinical Use After Myocardial Infarction. Stem Cells Transl Med. 2019; 8(8):822-832.

Embodiments of the invention are set out in the following numbered paragraphs:
1. An in vitro method for selecting CD34+ cells comprising:
   (i) determining the amount of VEGF expressed by a population of CD34+ cells, and
   (ii) selecting CD34+ cells based on the amount of VEGF expressed by the population.
2. The method of paragraph 1, wherein the CD34+ cells are selected if the amount of VEGF expressed by the cells in a culture medium is at least about 150 μg/ml.
3. The method of paragraph 1, wherein the CD34+ cells are selected if the amount of VEGF expressed by the cells is at least about 1 fg/cell.
4. An in vitro method for selecting CD34+ cells comprising:
   (i) detecting one or more microRNAs expressed by a population of CD34+ cells and/or contained in their exosomes, and
   (ii) selecting CD34+ cells based on the one or more microRNAs expressed by the population.
5. An in vitro method for selecting CD34+ cells comprising:
   (i) detecting one or more microRNAs expressed by a population of CD34+ cells and/or contained in their exosomes and determining the amount of VEGF expressed by a population of CD34+ cells, and
   (ii) selecting CD34+ cells based on the one or more detected microRNAs and the amount of VEGF expressed.
6. The method of paragraph 4 or paragraph 5, wherein the CD34+ cells are selected if the expression of one or more or each of miR126, miR130a, miR21, miR26a, miR378a, miR146a, miR199a, miR590 and miR133a is detected.
7. The method of paragraph 6, wherein the CD34+ cells are selected if the expression of one or more or each of miR126, miR130a, miR21, miR26a, and miR378a is detected.
8. The method of paragraph 6 or paragraph 7, wherein the CD34+ cells are selected if the expression of one or more or each of miR21, miR26a, and miR378a is detected.
9. The method of paragraph 6, wherein the CD34+ cells are selected if the expression of miR146a and/or miR21 is detected.
10. The method of paragraph 6, wherein the CD34+ cells are selected if the expression of miR199a and/or miR590 is detected.
11. The method of any preceding paragraph, wherein the CD34+ cells are human CD34+ cells.
12. The method of any preceding paragraph, wherein the CD34+ cells are autologous or allogeneic CD34+ cells.
13. The method of any preceding paragraph, further comprising expanding a population of CD34+ cells before and/or after steps (i) or (ii).
14. The method of paragraph 13, wherein the cells are expanded for 9 days.
15. The method of paragraph 13 or paragraph 14, wherein the expanding comprises one or more of;
   expanding at 37° C.,
   expanding in a 5% CO₂ controlled atmosphere, and/or
   expanding in a culture medium comprising cytokines such as interleukin 6 (IL6), interleukin 3 (IL3), Stem Cell Factor, ThromboPoietin, and/or Fms-Like Tyrosin kinase 3 Ligand.
16. The method of any preceding paragraph, wherein the amount of VEGF is determined by ELISA or automated ELISA (ELLA).
17. The method of any one of paragraphs 1 to 16, wherein the amount of VEGF is determined by mass spectrometry.
18. The method of any one of paragraphs 1 to 16, wherein the amount of VEGF is determined by radioimmunoassays.
19. The method of any one of paragraphs 1 to 16, wherein the amount of VEGF is determined by multiplex assay.
20. The method of any preceding paragraph, further comprising collecting, centrifuging and/or purifying the selected cells, optionally wherein the purifying is by immunoselection.
21. The method of any preceding paragraph, wherein the cells are for use in treatment.
22. An isolated population of CD34+ cells selected by the method of any one of paragraphs 1 to 21.
23. An isolated population of CD34+ cells selected by the method of any one of paragraphs 1 to 21, for use in therapy.
24. An isolated population of CD34+ cells selected by the method of any one of paragraphs 1 to 21, for use in the treatment of myocardial infarction.
25. The isolated population of CD34+ cells according to paragraph 23 or 24, wherein administration of the CD34+ cells to a subject reduces NT-proBNP in the subject.

1. An in vitro method for selecting CD34+ cells comprising:
(i) determining the amount of VEGF expressed by a population of CD34+ cells, and
(ii) selecting CD34+ cells if the amount of VEGF expressed by the population of CD34+ cells in the cell culture medium or supernatant of a CD34+ cell culture is at least about 100 μg/ml.

2-15. (canceled)

16. An in vitro method for obtaining CD34+ cells comprising:
(i) expanding a population of CD34+ cells, wherein said expanding comprises expanding for 5 to 12 days, at 37° C., in a 5% CO₂ controlled atmosphere and in a culture medium comprising cytokines selected from interleukin 6 (IL6), interleukin 3 (IL3), Stem Cell Factor, ThromboPoietin, and/or Fms-Like Tyrosin kinase 3; and
(ii) selecting CD34+ cells expressing an amount of VEGF in the cell culture medium or supernatant of a CD34+ cell culture, of at least about 100 μg/ml.

17. The method of claim 16, wherein said selected cells express VEGF at a concentration of at least about 150 pg/mL or at least about 200 pg/mL in the cell culture medium or supernatant of a CD34+ cell culture and/or wherein said cells express VEGF at an amount of at least about 1 fg/cell, or at least about 2 fg/cell or at least about 3 fg/cell.

18. The method of claim 16, further comprising:
(i) detecting one or more microRNAs expressed by a population of CD34+ cells and/or contained in their exosomes, and
(ii) selecting CD34+ cells based on the expression of one or more microRNAs expressed by the population.

19. The method of claim 16, wherein said cells express one or more or each of miR126, miR130a, miR21, miR26a, miR378a, miR146a, miR199a, miR590 and miR133a.

20. The method of claim 16, wherein the amount of VEGF is determined by ELISA, automated ELISA (ELLA), mass spectrometry, radioimmunoassays or multiplex assay.

21. The method of claim 16, further comprising collecting, centrifuging, purifying, resuspending and/or conditioning the selected cells, optionally wherein the purifying is by immunoselection.

22. The method of claim 16, further comprising processing the cells to form a composition suitable for therapeutic use.

23. An isolated population of CD34+ cells obtained by the method of claim 16.

24. An isolated population of expanded CD34+ cells, wherein said cells are obtained from a whole blood sample of a human donor subject and are expanded:
for 5 to 12 days;
at 37° C.;
in a 5% CO₂ controlled atmosphere; and
in a culture medium comprising cytokines selected from interleukin 6 (IL6), interleukin 3 (IL3), Stem Cell Factor, ThromboPoietin, and/or Fms-Like Tyrosin kinase 3,
wherein said cells express VEGF at a concentration of at least 100 pg/mL in the cell culture medium or supernatant of a CD34+ cell culture.

25. The isolated population of expanded CD34+ cells of claim 24, wherein said cells express VEGF at a concentration of at least 150 pg/mL or at least 200 pg/mL in the cell culture medium or supernatant of a CD34+ cell culture and/or wherein said cells express VEGF at an amount of at least about 1 fg/cell, or at least about 2 fg/cell or at least about 3 fg/cell.

26. The isolated population of expanded CD34+ cells of claim 24, wherein said cells express one or more or each of miR126, miR130a, miR21, miR26a, miR378a, miR146a, miR199a, miR590 and miR133a.

27. An isolated population of CD34+ cells according to claim 23, for use in therapy.

28. An isolated population of CD34+ cells according to claim 23, for use in the treatment of myocardial infarction or any other ischemic lesion.

29. An isolated population of CD34+ cells according to claim 24, for use in therapy.

30. An isolated population of CD34+ cells according to claim 24, for use in the treatment of myocardial infarction or any other ischemic lesion.

31. The isolated population of CD34+ cells according to claim 27, wherein administration of the CD34+ cells to a subject reduces NT-proBNP and LV remodelling and improves viability of myocardium in the subject.

32. The isolated population of CD34+ cells according to claim 29, wherein administration of the CD34+ cells to a subject reduces NT-proBNP and LV remodelling and improves viability of myocardium in the subject.


SEQUENCE LISTING (18 sequences)

SEQ ID NO: 1 - RNA, 22 nt, synthetic construct
  source 1..22: mol_type = other RNA
  ncRNA 1..22: ncRNA_class = miRNA
tcgtaccgtgagtaataatgcg

SEQ ID NO: 2 - RNA, 22 nt, synthetic construct
  source 1..22: mol_type = other RNA
  ncRNA 1..22: ncRNA_class = miRNA
cagtgcaatgttaaaagggcat

SEQ ID NO: 3 - RNA, 22 nt, synthetic construct
  source 1..22: mol_type = other RNA
  ncRNA 1..22: ncRNA_class = miRNA
tagcttatcagactgatgttga

SEQ ID NO: 4 - RNA, 22 nt, synthetic construct
  source 1..22: mol_type = other RNA
  ncRNA 1..22: ncRNA_class = miRNA
ttcaagtaatccaggataggct

SEQ ID NO: 5 - RNA, 22 nt, synthetic construct
  source 1..22: mol_type = other RNA
  ncRNA 1..22: ncRNA_class = miRNA
actggacttggagtcagaaggc

SEQ ID NO: 6 - RNA, 22 nt, synthetic construct
  source 1..22: mol_type = other RNA
  ncRNA 1..22: ncRNA_class = miRNA
tgagaactgaattccatgggtt

SEQ ID NO: 7 - RNA, 22 nt, synthetic construct
  source 1..22: mol_type = other RNA
  ncRNA 1..22: ncRNA_class = miRNA
acagtagtctgcacattggtta

SEQ ID NO: 8 - RNA, 21 nt, synthetic construct
  source 1..21: mol_type = other RNA
  ncRNA 1..21: ncRNA_class = miRNA
taattttatgtataagctagt

SEQ ID NO: 9 - RNA, 22 nt, synthetic construct
  source 1..22: mol_type = other RNA
  ncRNA 1..22: ncRNA_class = miRNA
tttggtccccttcaaccagctg

SEQ ID NO: 10 - RNA, 21 nt, synthetic construct
  source 1..21: mol_type = other RNA
  ncRNA 1..21: ncRNA_class = miRNA
cattattacttttggtacgcg

SEQ ID NO: 11 - RNA, 22 nt, synthetic construct
  source 1..22: mol_type = other RNA
  ncRNA 1..22: ncRNA_class = miRNA
gctcttttcacattgtgctact

SEQ ID NO: 12 - RNA, 23 nt, synthetic construct
  source 1..23: mol_type = other RNA
  ncRNA 1..23: ncRNA_class = miRNA
cctcctgactccaggtcctgtgt

SEQ ID NO: 13 - RNA, 23 nt, synthetic construct
  source 1..23: mol_type = other RNA
  ncRNA 1..23: ncRNA_class = miRNA
cccagtgttcagactacctgttc

SEQ ID NO: 14 - RNA, 22 nt, synthetic construct
  source 1..22: mol_type = other RNA
  ncRNA 1..22: ncRNA_class = miRNA
gagcttattcataaaagtgcag

SEQ ID NO: 15 - RNA, 22 nt, synthetic construct
  source 1..22: mol_type = other RNA
  ncRNA 1..22: ncRNA_class = miRNA
agctggtaaaatggaaccaaat

SEQ ID NO: 16 - RNA, 21 nt, synthetic construct
  source 1..21: mol_type = other RNA
  ncRNA 1..21: ncRNA_class = miRNA
caacaccagtcgatgggctgt

SEQ ID NO: 17 - RNA, 22 nt, synthetic construct
  source 1..22: mol_type = other RNA
  ncRNA 1..22: ncRNA_class = miRNA
cctattcttggttacttgcacg

SEQ ID NO: 18 - RNA, 22 nt, synthetic construct
  source 1..22: mol_type = other RNA
  ncRNA 1..22: ncRNA_class = miRNA
cctctgaaattcagttcttcag